(19) United States
(12) Patent Application Publication
(10) Pub. No.: US 2003/0097683 A1
(43) Pub. Date: May 22, 2003

(54) Single-component RNA vectors derived from a virus and containing an intervening sequence between the cap and the 5' end and able to replicate in a host plant cell within a host plant

(75) Inventors: John A. Lindbo, Vacaville, CA (US); Gregory P. Pogue, Vacaville, CA (US); Thomas H. Turpen, Vacaville, CA (US)

Correspondence Address: QUINE INTELLECTUAL PROPERTY LAW GROUP, P.C., P O BOX 458, ALAMEDA, CA, 94501, US

(73) Assignee: Large Scale Biology Corporation, Vacaville, CA

(21) Appl. No.: 10/200,051

(22) Filed: Jul. 18, 2002

Related U.S. Application Data
(63) Continuation-in-part of application No. 09/949,316, filed on Sep. 7, 2001.
Application No. 09/949,316 is a continuation of application No. 09/502,711, filed on Feb. 11, 2000, now Pat. No. 6,300,134.
Application No. 09/502,711 is a continuation-in-part of application No. 09/359,301, filed on Jul. 21, 1999, now Pat. No. 6,426,185.
Application No. 09/502,711 is a continuation-in-part of application No. 09/359,305, filed on Jul. 21, 1999.
Application No. 09/359,305 is a continuation-in-part of application No. 09/232,170, filed on Jan. 15, 1999, now abandoned.
Application No. 09/232,170 is a continuation-in-part of application No. 09/008,186, filed on Jan. 16, 1998.

(51) Int. Cl. A01H 1/00; C07H 21/02; C12N 7/00

(52) U.S. Cl. 800/280; 435/235.1; 536/23.1

(57) ABSTRACT

This invention is directed to a plus strand RNA viral vector for transformation of a host organism with a foreign RNA, and expression of said foreign RNA. The foreign RNA is inserted into an infective RNA viral segment containing cis-acting viral replication elements, and allowed to infect the host organism. The RNA vector is modified to obtain infectivity by including an intervening sequence between the cap and the 5′ end. The modified RNA is able to tolerate the exogenous RNA segment without disrupting the replication of the modified RNA, in the absence of a trans-acting viral replication element in a single component plant virus host cell.

This application is a continuation-in-part of U.S. patent application Ser. No. 09/949,316, which is a continuation of U.S. Pat. No. 6,300,134 issued Oct. 9, 2001, which is a continuation-in-part of U.S. patent application Ser. Nos. 09/359,301 and 09/359,305, filed Jul. 21, 1999, which are continuations-in-part of U.S. patent application Ser. No. 09/232,170, filed Jan. 15, 1999, which is a continuation-in-part of U.S. patent application Ser. No. 09/008,186, filed Jan. 16, 1998. The above parent applications are incorporated herein by reference.

### FIELD OF THE INVENTION

This invention relates to the field of plant viruses, more particularly to plus-sense RNA plant virus, and to modifications, made according to the teachings herein, which permit insertion of an exogenous RNA segment into the viral genome. The recombinant plant viral nucleic acid construct derived from insertion of an exogenous RNA segment into the viral genome can then be introduced into a host cell in order to modify the cell.

### BACKGROUND OF THE INVENTION

RNA viruses whose genome is composed of a single RNA strand capable of replication in the cytoplasm of a host by direct RNA replication are widespread, many varieties of which are known to infect plants. Such viruses are sometimes termed “(+) strand RNA viruses” since the infective RNA strand, that normally found encapsidated in the virus particle, is a messenger-sense strand, capable of being directly translated, and also capable of being replicated under the proper conditions by a direct process of RNA replication. Viruses belonging to this group include “single component (+) strand RNA viruses”, which replicate in the absence of trans-acting viral replication elements. These viruses may include, but are not limited to any of the representatives of the following virus groups, Carlavirus, Closteroviridae, Luteoviridae, Potexvirus, Potyviridae, Tombusviridae, Tobamovirus and Tymovirus. (Similar viruses, which in the host cell produce a trans-acting replication element, are not included in this group.) In these cases, the entire virus genome is contained within a single RNA molecule, while in the multicomponent RNA plant viruses, the total genome of the virus consists of two or more distinct RNA segments, each separately encapsidated. For general review, see General Virology, S. Luria and J. Darnell; Plant Virology 2nd ed., R. E. F. Matthews, Academic Press (1981). For a general review of (+) strand RNA replication, see Davies and Hull (1982) J. Gen. Virol. 61:1.

Despite the well-documented diversity between virus groups, recent studies have shown striking similarities between the proteins, which function in RNA replication. Sequence homologies have been reported between the cowpea mosaic virus, poliovirus and foot-and-mouth disease virus, (Franssen, H. (1984) EMBO Journal 3,855). Sequence homologies have been reported between non-structural proteins encoded by alfalfa mosaic virus, brome mosaic virus and tobacco mosaic virus, Haseloff, J. et al. (1984), Proc. Nat. Acad. Sci. USA 81, 4358, and between non-structural proteins encoded by sindbis virus, Ahlquist, P. et al. (1985) J. Virol. 53, 536. Evidence of such substantial homology in proteins related to the replication functions indicate that the viruses share mechanistic similarities in their replication strategies and may actually be evolutionarily related. Ahlquist et al., in U.S. Pat. No. 5,500,360 made modifications to the genomic RNA of a (+) strand RNA virus of a multipartite Brome mosaic virus. The modified RNA was used to transfer a desired RNA segment into a targeted host plant protoplast, and to replicate that segment and express its function within the host protoplast.

In contrast to the Brome mosaic virus (BMV), the tobacco mosaic virus (TMV) is one member of a class of plant viruses characterized by a single RNA genome. The genetic material of the virus is RNA, and the total genetic information required for replication and productive infection is contained in one discrete RNA molecule. Infection of a host plant cell occurs when the single RNA component of the viral genome has infected the cell, for example by exposing a plant to a virus preparation. Infection may also be achieved by exposing a plant cell or protoplast to a virus preparation. TMV does not require coat protein for infection. The RNA component is both necessary and sufficient for replication and productive infection. The TMV genome is a single messenger-sense RNA. The term “messenger-sense” denotes that the viral RNAs can be directly translated to yield viral proteins, without the need for an intervening transcription step.

Complete cDNA copies of the genetic component of TMV have been cloned. Construction of a library of subgenomic cDNA clones of TMV has been described in Dawson et al., Proc. Natl. Acad. Sci. USA 83:1832-1836 (1986) and Ahlquist et al., Proc. Natl. Acad. Sci. USA 81:7066-7070 (1984). Several examples of TMV transcription vectors are described below. DNA from each of the TMV cDNA-containing plasmids can be cleaved. The linear DNA thus produced can be transcribed in vitro in a reaction catalyzed by RNA polymerase. A T7 promoter in the transcription vector allows RNA synthesis to initiate at the 5′ terminus of each TMV sequence, and transcription continues to the end of the DNA template. The 5′ terminus of tobacco mosaic virus (TMV) RNA, was identified as m.sup.7 G.sup.5′ ppp.sup.5′ Gp. Zimmem, D., Nucleic Acid Res. 2:1189-1201 (1975). Keith, J. and fraenkel-Conrat, H. FEBS Lett. 57:31-33 (1975). Ahlquist, U.S. Pat. No. 5,500,360, working with Brome mosaic virus, reported that when transcription is carried out in the presence of a synthetic cap structure, m.sup.7 GpppG, as described by Contreras, R., et al. Nucleic Acids Res. 10:6353, (1982), RNA transcripts are produced with the same capped 5′ ends as authentic BMV RNAs. Ahlquist concluded that these RNAs are active messengers in in vitro translation systems and direct production of proteins with the same electrophoretic mobilities as those translated from authentic BMV RNAs. However, Ahlquist found that, “if the cap analog was omitted during in vitro transcription, no infection was detected, even if inoculum concentration was increased 20-fold.” Further, Ahlquist taught only a viral vector having “no extraneous nonviral sequences between the cap and the 5′ terminus of the viral sequence.” In Ahlquist's work on BMV, U.S. Pat. No. 5,500,360, a transcription vector was employed which preserved the exact 5′ terminal nucleotide sequence of viral RNA. It is now generally accepted that capping is necessary for infectivity and that no intervening sequence can be present between the cap and the 5′ ternminus of the viral sequence.

The work of Ahlquist leaves us with difficult problems to overcome if we are to obtain a workable viral vector or a commercially viable viral vector. One such problem is the cost of using capping structures and cap analogs. Another such problem is that multipartite viral vectors are difficult to use relative to a single component viral vector. Multipartite viruses require more than one unit to infect and achieve replication in a host plant, and multipartite viruses require a trans acting replication element to achieve replication. No one has yet found a way to unite the multiple strands of a multipartite virus into an RNA molecule comprising the entire genome of a (+) strand RNA virus as suggested and claimed by Ahlquist.

Therefore, there is a need for a viral vector that can accept an intervening base or intervening sequence of bases between the cap and the 5′ terminus of the viral sequence and undergo transcription and replication. There is also a need for a viral vector that can undergo transcription and replication in the absence of a capping structure.

Here we teach solutions to the problem by demonstrating:

1. Infection of a host and replication of a viral vector in vivo in the presence of a base or a sequence of bases placed 5′ to the origin of replication in the absence of a capping structure or cap analog.

2. Infection of a host and replication of a viral vector in vivo in the absence of a capping structure or a cap analog, and in the absence of a base or a sequence of bases placed 5′ to the origin of replication.

3. Infection of a host and replication of a viral vector in vivo in the presence of an intervening base or an intervening sequence of bases placed 5′ to the origin of replication and in the presence of a capping structure or cap analog.

The viral vectors demonstrated here have utility in discovery the function of genes, and in production of therapeutic proteins.

### SUMMARY OF THE INVENTION

The For the sake of brevity, the term “RNA virus” is used herein to mean (+) strand replicating RNA viruses. Most single component RNA viruses have the advantage over multicomponent RNA viruses of having a single RNA structure. Because they have a single RNA structure, the function of an exogenous RNA segment can be expressed in a host cell in the absence of a trans-acting replication element. Further, the single component RNA virus does not express the 3a movement gene that is indigenous to the Brome mosaic virus.

The invention is based on the discovery that leaving out the capping structure so that the virus transcript is uncapped can modify the 5′ end of a single component RNA viral vector. The invention is also based on the discovery that the 5′ end of a single component RNA viral vector can be modified by inserting a base or a sequence of bases ahead of the 5′ terminus of the viral sequence. The invention is also based on the discovery that the 5′ end of a single component RNA viral vector can be modified by inserting an intervening base or an intervening sequence of bases between the cap and the 5′ terminus of the viral sequence. The genome of a virus, modified in each of these three ways, can be further modified to include an exogenous RNA segment. The further modified RNA can be introduced into a host cell where it will replicate and express the exogenous RNA segment. The recipient cell is thereby phenotypically transformed and may contribute to a genotypically transformed organism, as well.

Phenotypically transformed plants and plant cells can be modified in vivo, in planta, in tissue culture, in cell culture or in the form of protoplasts. The exemplified embodiment of the invention is useful for producing phenotypically transformed plants under field or greenhouse growth conditions. Traits desirable for introduction in this manner include, but are not limited to, pest resistance, pathogen resistance, herbicide tolerance or resistance, modified growth habit and modified metabolic characteristics, such as the production of commercially useful peptides or pharmaceuticals in plants. The modifications can be applied at any time during the growth cycle, depending on the need for the trait. For example, resistance to a pest could be conferred only if the crop were at risk for that pest, and at the time when the crop was most likely to be affected by the pest. Other traits can be used to enhance secondary properties, for example to increase the protein content of post-harvest forage. Any plant variety susceptible to infection by a single component RNA virus can be phenotypically transformed. The choice of virus and the details of modification will be matters of choice depending on parameters known and understood by those of ordinary skill in the art. Other uses for cells and organisms phenotypically or genotypically modified by means of a modified RNA derived from an RNA virus will be readily apparent to those skilled in the art, given a wide range of RNA viruses to modify and a wide range of susceptible host cell types. Other uses for transformed animal cells, bacterial cells and the like can be readily envisioned but are not demonstrated here.

Generally, the steps of a process for phenotypically transforming a cell or organism are:

forming a full-length cDNA transcript of the RNA virus;

cloning the cDNA in a transcription vector;

modifying the cDNA by inserting a non-viral DNA segment in a region able to tolerate such insertion without disrupting RNA replication thereof;

transcribing the modified cDNA corresponding to the RNA component of the single component virus;

infecting virus-susceptible protoplasts, cells, tissues or whole plants with transcribed RNA, either in solution or encapsidated, of the modified RNA comprising messenger-sense RNA containing an exogenous RNA segment.

From this point, the steps to be followed will vary, depending on the type of material infected and the route of infection. Protoplasts, cells and tissues of plants can be propagated vegetatively, regenerated to yield whole plants by means of any technique suitable to the particular plant variety infected, and transplanted to the field. Whole plants can be infected in situ. Infected plants and plant cells can produce many copies per cell of the modified viral RNA containing the exogenous RNA segment. If desired and if suitably inserted, by means of principles and processes known in the art, the exogenous RNA segment can be caused to carry out a function within the cell. Such a function could be a coding function, translated within the cell to yield a desired peptide or protein, or it could be a regulatory function, increasing, decreasing, and turning on or off the expression of certain genes within the cell. In principle, any function, which a segment of RNA is capable of providing, can be expressed within the cell. The exogenous RNA segment thus expressed confers a new phenotypic trait to the transformed organism, plant, cells, protoplasts or tissues.

The invention is exemplified herein by the modification of TMV RNA to contain a structural gene encoding green fluorescent protein (GFP) and the phenotypic modification of Nicotiana plants and protoplasts therewith, yielding plants and protoplasts synthesizing GFP. The data presented herein are believed to represent the first instance of phenotypic modification of a cell by means of an RNA virus which is uncapped and which has no base at the 5′ end of the uncapped viral sequence.

The data presented herein are believed to represent the first instance of phenotypic modification of a cell by means of an RNA virus which is uncapped and which has a single base or a sequence of bases at the 5′ end of an uncapped viral sequence. The data presented herein are believed to represent the first instance of phenotypic modification of a cell by means of an RNA virus, which contains an intervening base, or intervening sequence of bases between the cap and the 5′ end of the viral sequence.

The present invention involves a capped RNA molecule capable of replication in the cytoplasm of a host plant cell located in a host plant. It is also capable of local spread in a host plant, and also capable systemic spread in a host plant. The Capped RNA molecule of the present invention comprises:

a) a cis-acting viral replication element obtained from a single-component (+) strand RNA plant virus, the plant virus capable of replication, local and systemic spread in a host plant;

b) a sequence of one to three intervening bases located between a cap and the 5′ terminus of the viral sequence; and

c) an exogenous RNA segment capable of expressing its function in a host plant cell; the exogenous RNA segment being located in a region of said capped RNA molecule able to tolerate said exogenous RNA segment without disrupting replication of said capped RNA molecule, wherein said uncapped RNA molecule is capable of replication in the absence of a trans-acting viral replication element in the host plant cell.

According to the present invention the exogenous RNA segment codes for a peptide or protein, and/or a structural protein. The exogenous RNA can be antisense RNA, or regulatory RNA, or RNA having catalytic properties. The cis-acting viral replication element can be derived from a tobacco mosaic virus or another single stranded RNA virus. The RNA molecule can be encapsidated with viral coat protein. A typical host for such a virus is Nicotiana sp.

The vector of the present invention typically goes through a cloning stage to form a DNA transcription vector. The DNA transcription vector comprises cDNA having one strand complementary to a capped RNA molecule, the capped RNA molecule being capable of replication, local and systemic spread in a host plant cell, and comprising:

a) a cis-acting viral replication element derived from a single-component (+) strand RNA plant virus, the plant virus capable of replication, local and systemic spread in the cytoplasm of a host plant cell, located a host plant; and

b) a sequence of one to three intervening bases located between the cap and the 5′ terminus of the viral sequence; and

c) an exogenous RNA segment capable of expressing its function in a host plant cell in a region of said capped RNA molecule able to tolerate said exogenous RNA segment without disrupting RNA replication of said capped RNA molecule; and wherein said capped RNA molecule is capable of replication in the absence of a trans-acting viral replication element in host plant cells.

A DNA transcription vector of the present invention include pBTI1037, pBTI SBS60, pBTI SBS60-29, pBTI1056, pBTI1057, and pBTI1056-GTN28.

The present invention includes a method of modifying a host plant phenotypically or biochemically, which method, comprise:

i) introducing into a host plant cell which is located within the host plant a capped RNA molecule capable of replication in the cytoplasm of a host plant cell located in a host plant, the capped RNA molecule, comprising:

a) a cis-acting viral replication element derived from a single-component (+) strand RNA plant virus, the plant virus capable of replication in host plant cells;

b) a sequence of one to three intervening bases located between the cap and the 5′ terminus of the viral sequence; and

c) an exogenous RNA segment segment capable of expressing its function in a host cell, said exogenous RNA segment being located in a region of said capped RNA molecule able to tolerate said exogenous RNA segment without disrupting RNA replication of said capped RNA molecule, and wherein said capped RNA molecule is capable of replication in the absence of a trans-acting viral replication element in host plant cells, whereby the exogenous RNA segment confers a detectable trait in the host plant.

This invention is also represented by (+) strand RNA virus from the group consisting of Carlavirus, Closteroviridae, Luteoviridae, Potexvirus, Potyviridae, Tombusviridae, Tymovirus and Tobamovirus. These viruses might be engineered with a second subgenomic promoter to control expression of the exogenous RNA.

### BRIEF DESCRIPTION OF THE DRAWINGS

FIG. 1 shows the sequence of pBTI 30BGFPc3 (p1037) (SEQ ID NO: 22).

FIG. 2 shows the sequence of pBTI SBS60 (SEQ ID NO: 23).

FIG. 3 shows the sequence of pBTI SBS60-29 (SEQ ID NO: 24).

FIG. 4 shows the sequence of pBTI1056 (SEQ ID NO: 25).

FIG. 5 shows the sequence of pBTI SBS5 (p1057) (SEQ ID NO: 26).

FIG. 6 shows the sequence of pBTI1056-GTN28 (SEQ ID NO: 27).

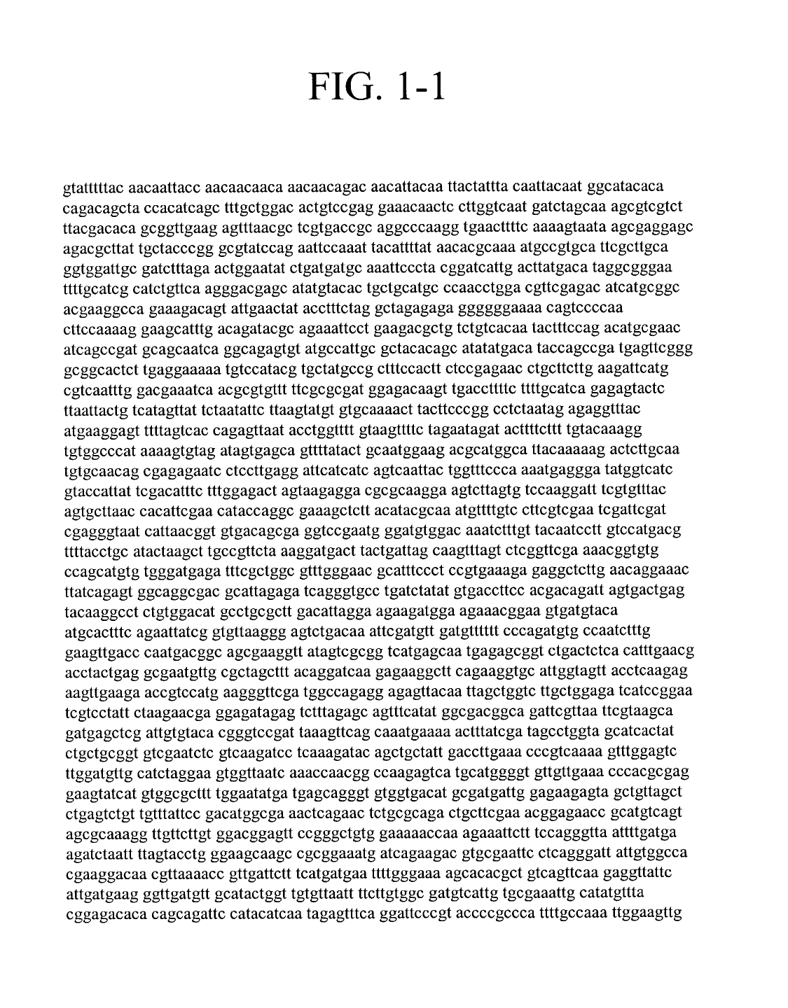

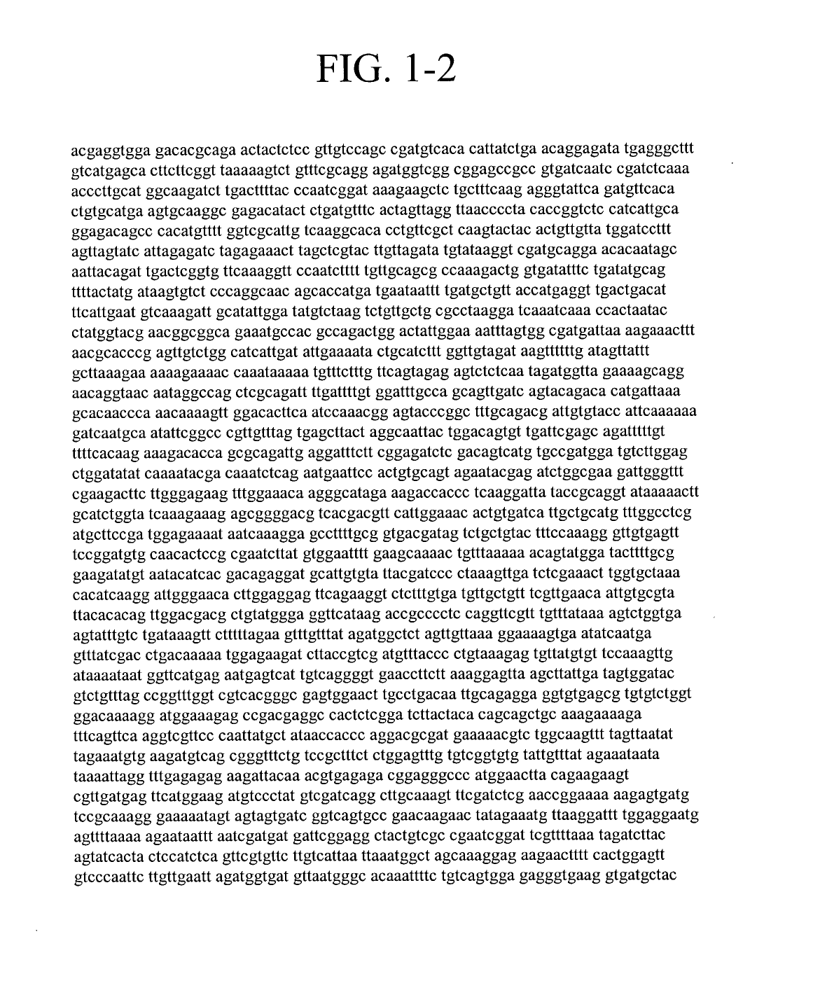

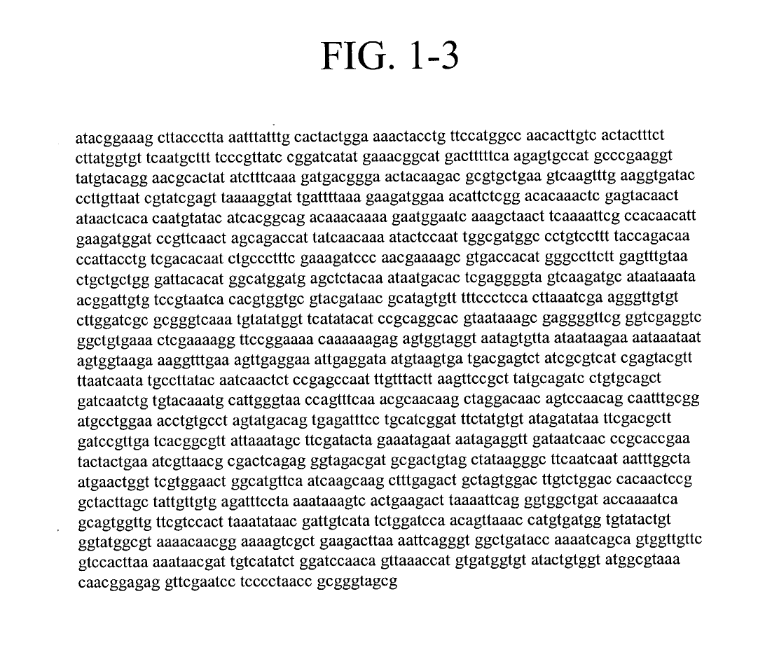

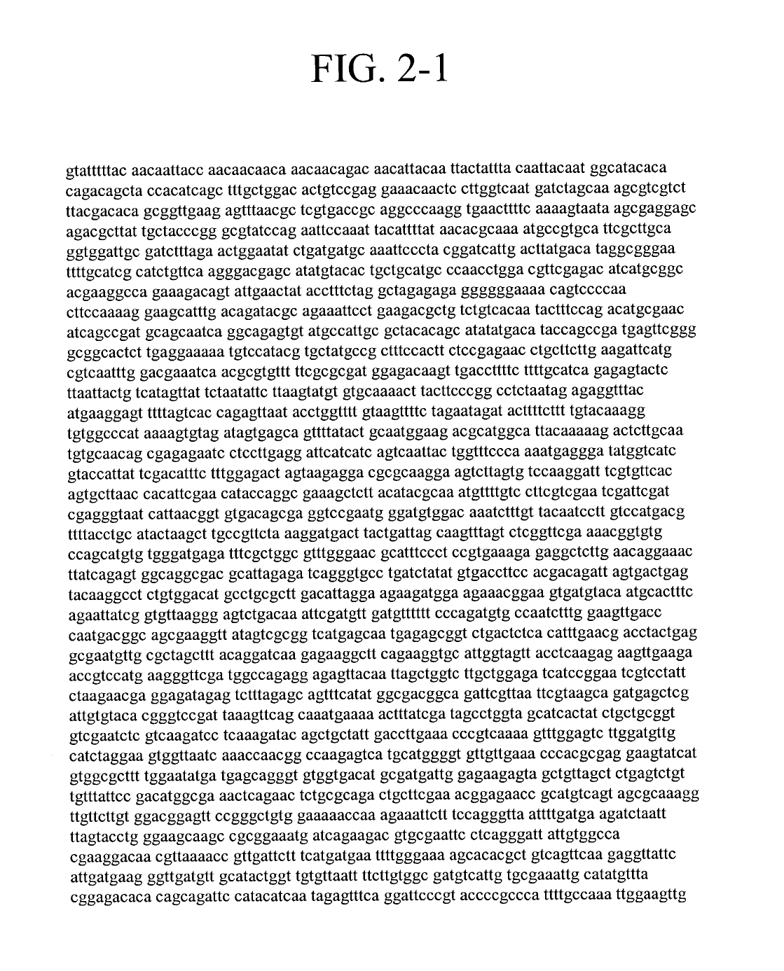

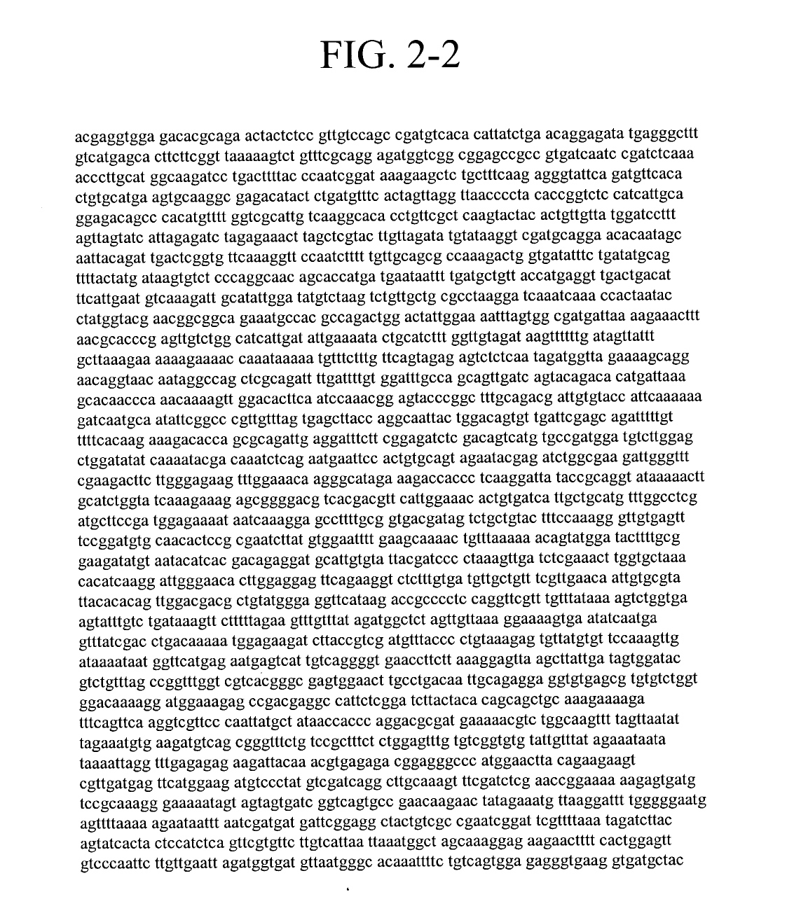

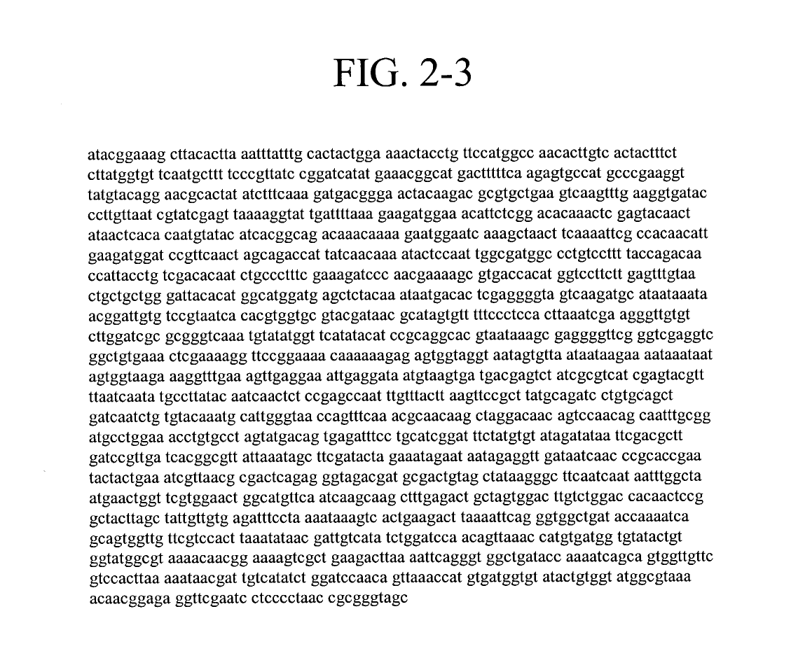

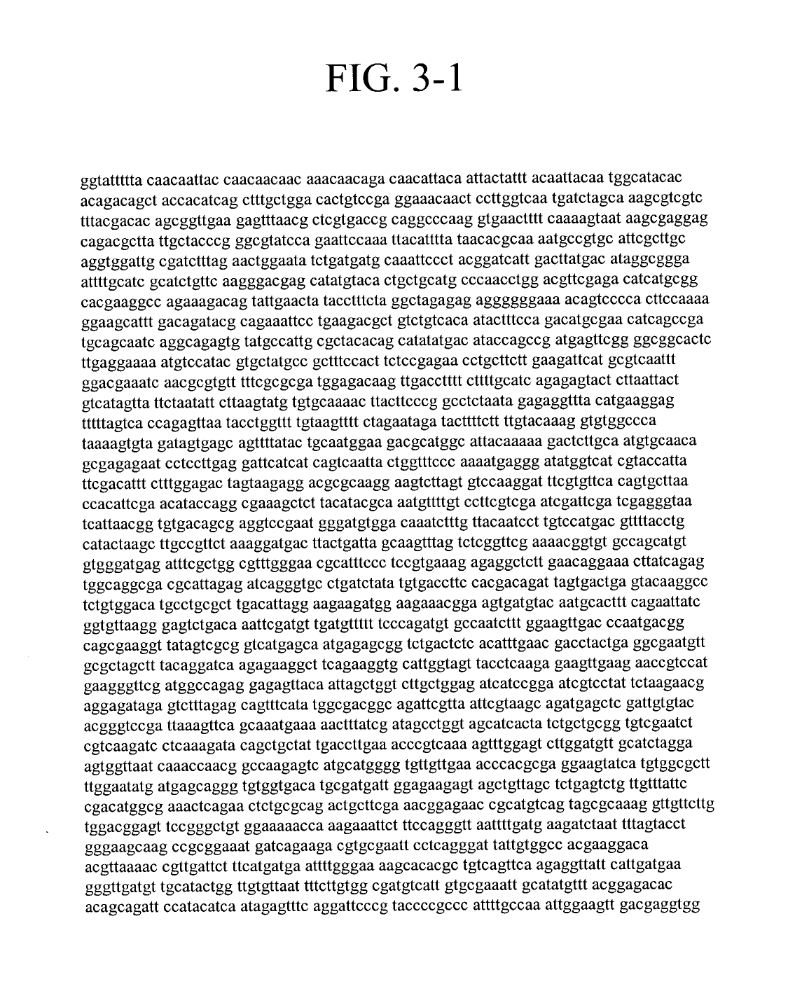

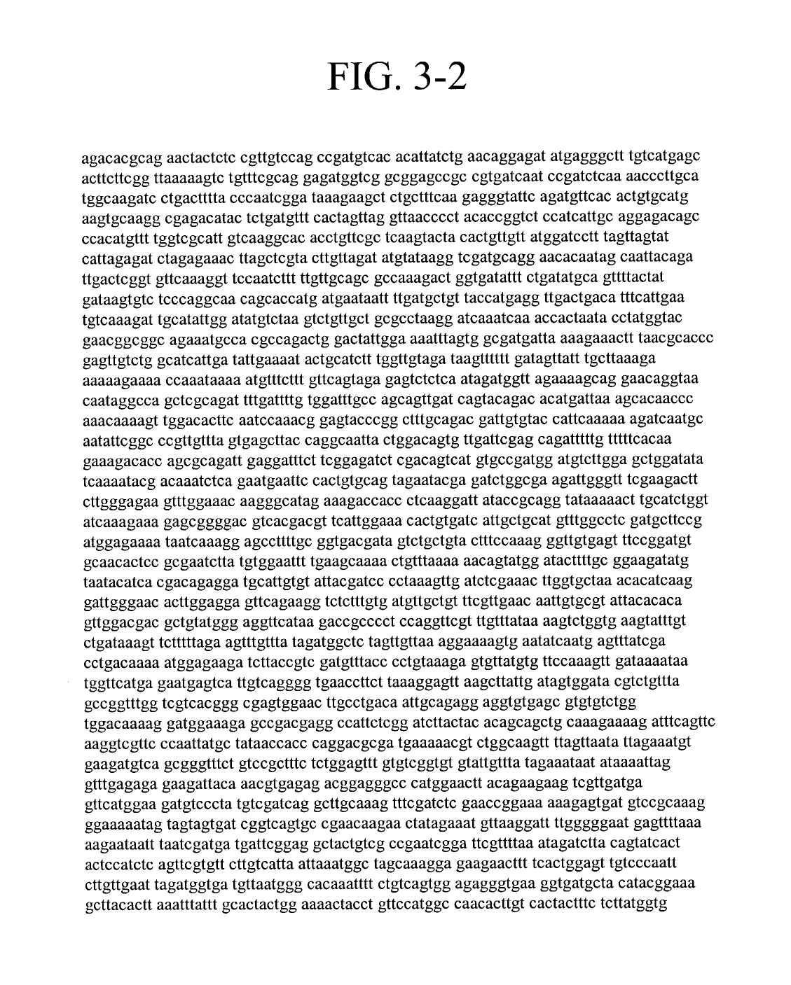

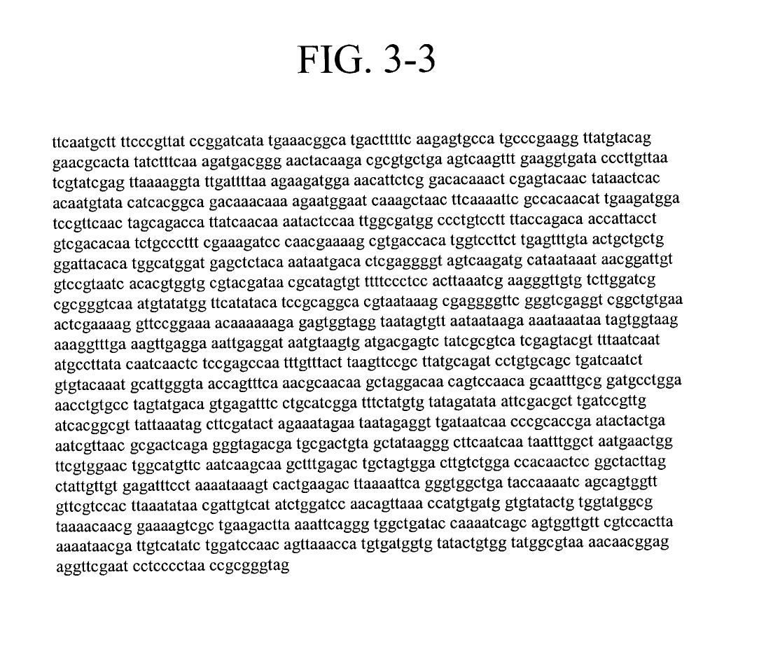

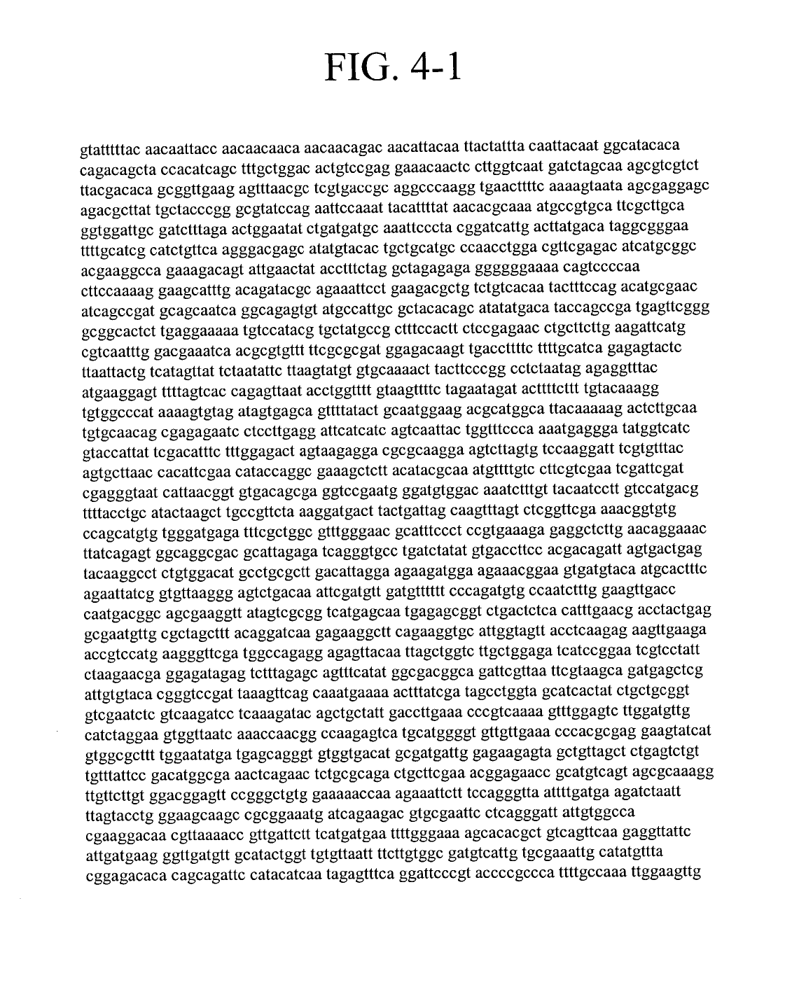

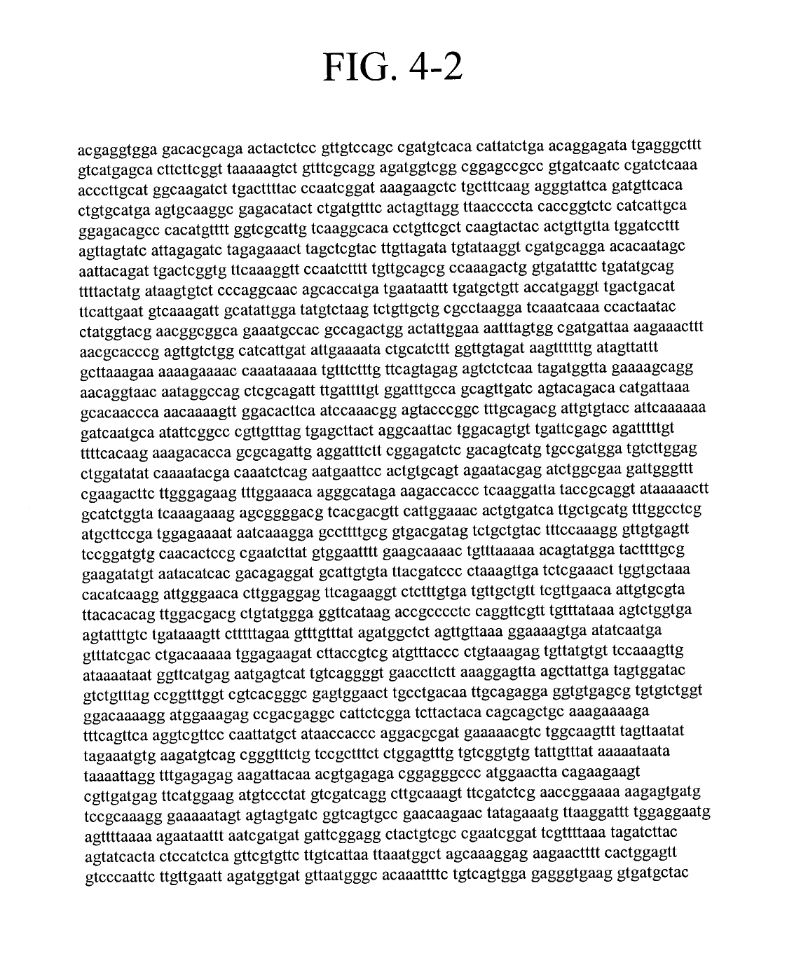

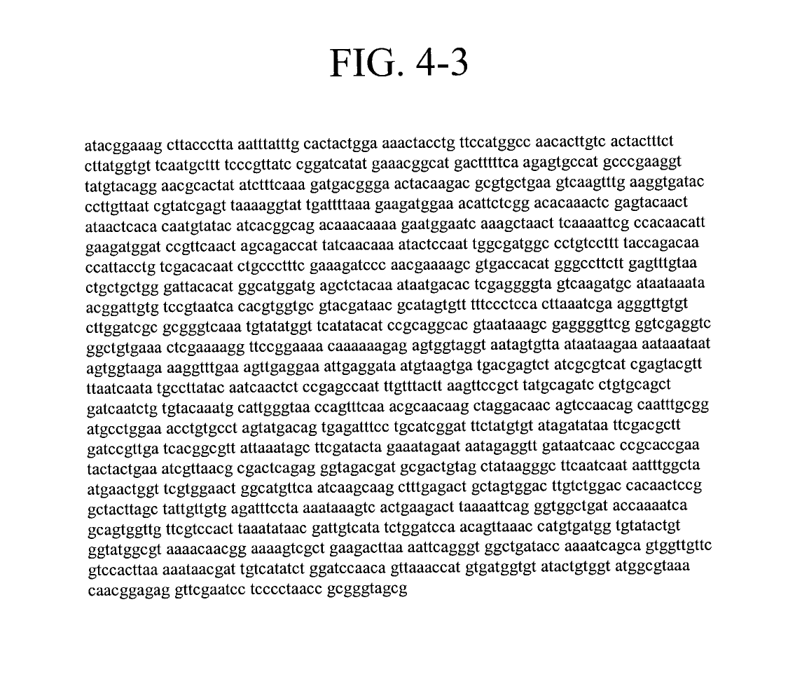

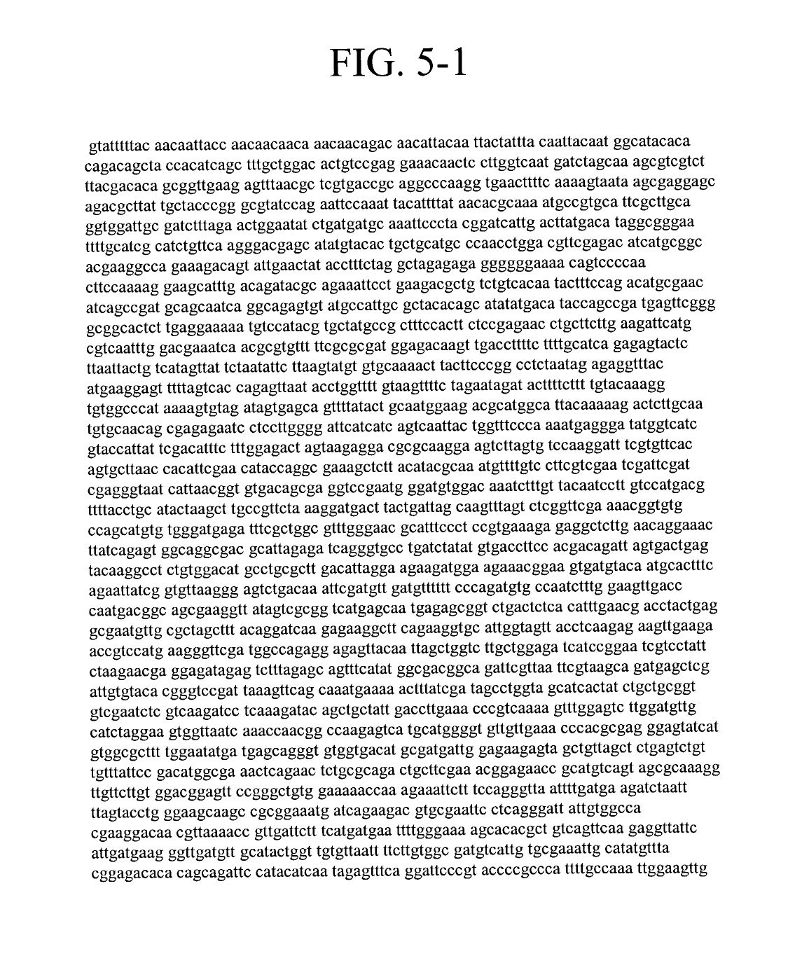

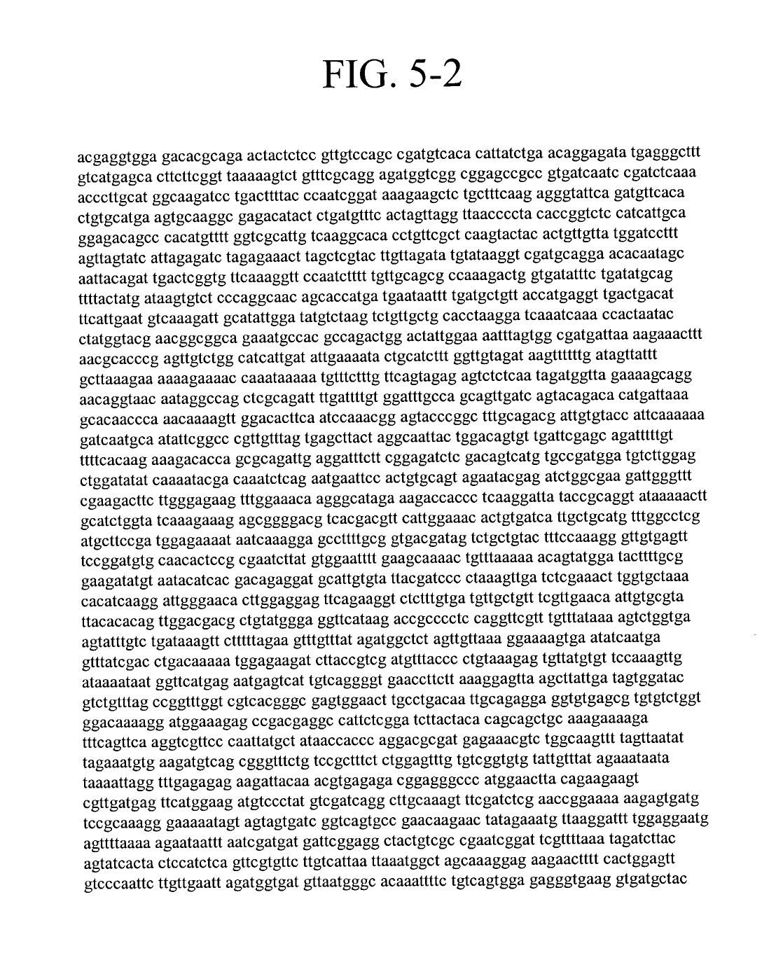

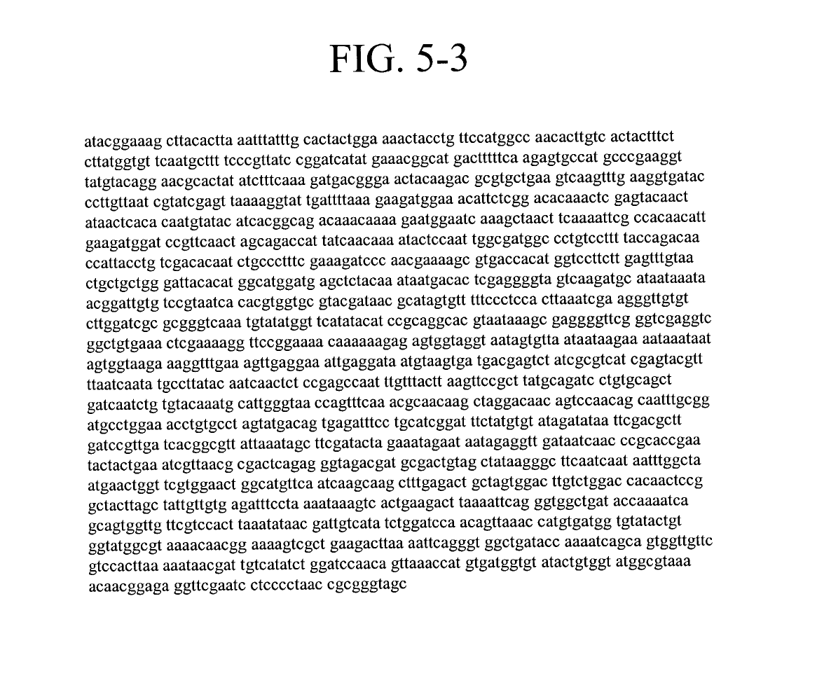

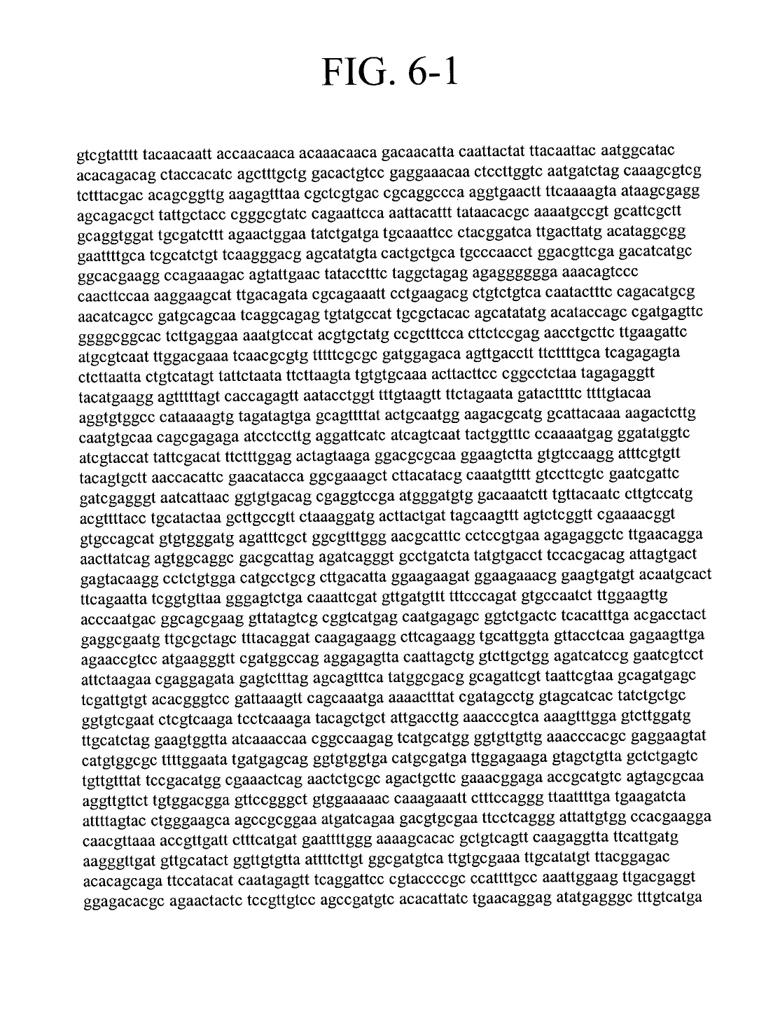

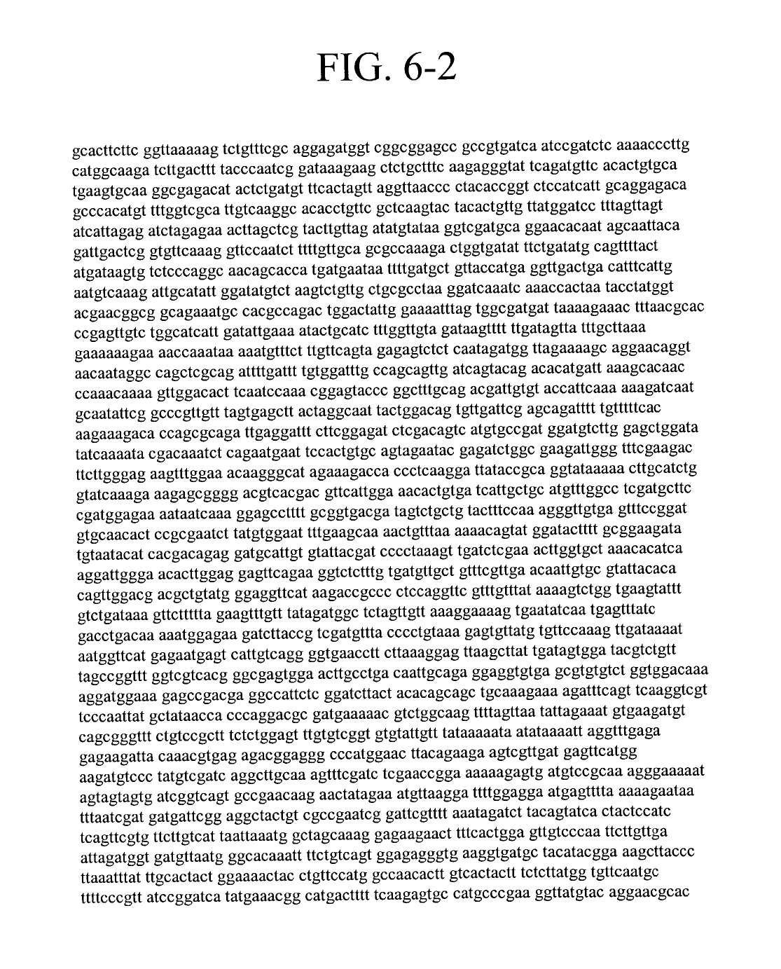

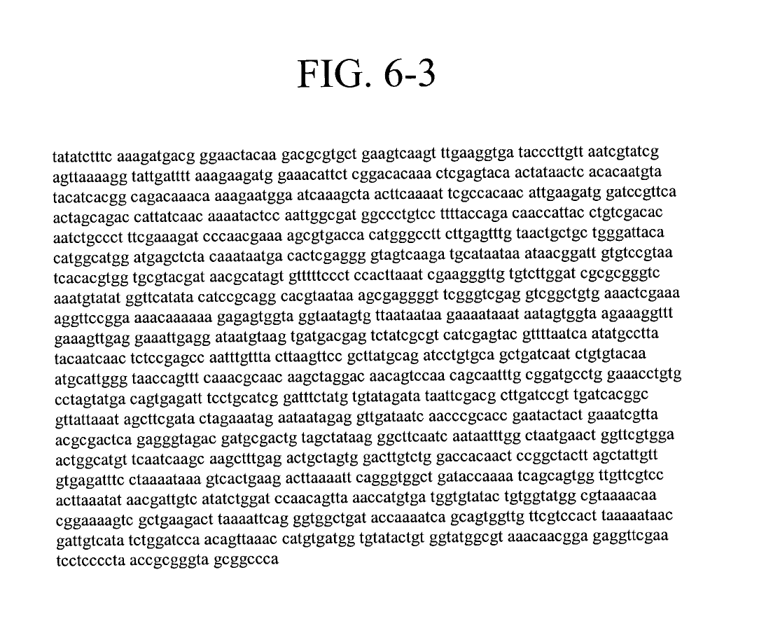

### DETAILED DESCRIPTION OF THE INVENTION

In order to facilitate understanding of the invention, certain terms used throughout are herein defined.

Assembly as used herein means The stage of replication during which all the structural components come together at one site in the cell and the basic structure of the virus particle is formed.

Base as used herein means adenine, thymidine, guanine, and cytosine, which in the form of a nucleotide can bond with other bases to form a nucleotide sequence. As used herein, a “base sequence” or a “sequence of bases” refers to a nucleotide sequence. The bases used herein are DNA bases, because all base or base sequence manipulations are performed on plasmid DNA prior to transcription. Base might be used interchangeably with “nucleotide”.

Cis-acting replication element as used herein means that portion of the RNA genome of an RNA virus, which must be present in cis, that is, present as part of each viral strand as a necessary condition for replication. Virus replication of a single component virus such as TMV has only cis-acting replication elements in its RNA. The cis-acting replication element is composed of one or more segments of viral RNA, which must be present on any RNA molecule that is to be replicated by RNA replication. The segment will most likely be the 5′ terminal portion of the viral RNA molecule, and may include other portions as well. As is demonstrated herein, using the example of TMV, portions of an RNA virus molecule may be modified without disrupting replication. A viral or non-viral gene may be inserted under the control of an additional subgenomic promoter. A viral or non-viral gene may be fused to a viral coat protein gene either under the control of the original subgenomic promoter of the viral coat protein gene, or under the control of an additional subgenomic promoter.

Derived from as used herein means the viral source of an RNA segment, which comprises part of the modified RNA. For example, for the modified RNAs described herein, substantial portions thereof are derived from TMV. The manner of deriving, whether by direct recombination at the RNA level, by transcription or by reverse transcription does not matter for the purpose of the invention. It is contemplated that modifications may be made within the cis-acting replication element and elsewhere for example to modify the rate or amount of replication that is obtained.

In the case of modified RNAs exemplified herein, a transcription vector was employed which, preserved the exact 5′ terminal nucleotide sequence of viral RNA, but a) left the capping structure off, or b) left the capping structure off and added a single base to the 5′ terminal nucleotide sequence of the viral cDNA, or c) left the capping structure off and added a sequence of bases to the 5′ terminal nucleotide sequence of the viral cDNA, or d) inserted a single intervening base between the capping structure and the 5′ terminal nucleotide sequence of the viral cDNA, or e) inserted an intervening sequence of bases between the cap and the 5′ terminal nucleotide sequence. The use of such a vector in transcribing viral RNA will be preferred if preservation of the exact nucleotide sequence at the 5′ end is desired. The use of such a vector in transcribing viral RNA will be preferred if the objective is to only remove the cap without further objectives with respect to the 5′ end of the virus. An RNA segment which has been derived from a given source virus may, but need not be, identical in sequence to that segment as it exists in the virus. It will be understood that a cis-acting replicating element derived from a given RNA virus may in some cases have minor modifications in the nucleotide sequence thereof without substantially interfering with RNA replication.

Exogenous RNA segment as used herein means a segment of RNA to be inserted into the virus RNA to be modified, the source of the exogenous RNA segment being different from the RNA virus itself. The source may be another virus, a living organism such as a plant, animal, bacteria, virus or fungus, the exogenous RNA may be a chemically synthesized RNA or it may be a combination of the foregoing. The exogenous RNA segment may provide any function that is appropriate and known to be provided by an RNA segment. Such functions include, but are not limited to, a coding function in which the RNA acts as a messenger RNA encoding a sequence which, translated by the host cell, results in synthesis of a peptide or protein having useful or desired properties. The RNA segment may also be structural, as for example in ribosomal RNA, it may be regulatory, as for example with small nuclear RNAs or anti-sense RNA, or it may be catalytic. A particularly interesting function is provided by anti-sense RNA, sometimes termed (−) strand RNA, which is in fact a sequence complementary to another RNA sequence present in the target cell which can, through complementary base pairing, bind to and inhibit the function of the RNA in the target cell. An exogenous RNA segment can be a complete or partial coding sequence.

Various aspects of the stages outlined in the Summary section can be modified as needed, depending upon specific aspects of the virus selected as the transforming agent and of the RNA segment to be inserted. For example, if the inserted gene is in the form of messenger-sense RNA to be directly translated by the transformed cell, the gene must be free of intervening, nontranslated sequences, such as introns. On the other hand, the inserted gene need not be a naturally occurring gene, but it may be modified, it may be a composite of more than one coding segment, or it may encode more than one protein. Combining insertions and deletions in order to control the total length or other properties of the modified RNA molecule may also modify the RNA. The inserted non-viral gene may be either prokaryotic or eukaryotic in origin as long as it is in a form, which can be directly translated by the translation machinery of the recipient cell. Eukaryotic genes containing introns within the coding sequence must therefore be inserted in the form of a cDNA copy of the eukaryotic messenger RNA encoding the gene. The inserted gene may contain its own translation start signals, for example, a ribosomal binding site and start (AUG) codon, or it may be inserted in a manner which takes advantage of one or more of these components preexisting in the viral RNA to be modified. Certain structural constraints must be observed to preserve correct translation of the inserted sequence, according to principles well understood in the art. For example, if it is intended that the exogenous coding segment be combined with an endogenous coding segment, the coding segment to be inserted must be inserted in reading frame phase therewith and in the same translational direction.

Gene expression as used herein means An important stage of viral replication at which virus genetic information is expressed: one of the major control points in replication.

Genome replication as used herein means The stage of viral replication at which the virus genome is copied to form new progeny genomes.

“Host plant cell located in a host plant” as used herein means a host plant cell that is part of a host plant organism, host plant tissue, or host plant organ.

Host as used herein means a host plant, host plant tissue or host cell within a host plant. In this context, a host plant is a multicellular organism having leaf and stem, and may be said to have plant cells, and plant tissues, plant organs or plant parts such as root, stem leaf, flower or seed. A host is capable of being infected by and capable of replicating a nucleic acid such as a plant viral nucleic acid.

Infection as used herein means the ability of a virus to transfer its nucleic acid to a host or introduce a viral nucleic acid into a host, wherein the viral nucleic acid is replicated, viral proteins are synthesized, and new viral particles assembled. In this context, the terms “transmissible” and “infective” are used interchangeably herein. The term is also meant to include the ability of a selected nucleic acid sequence to integrate into a genome, chromosome or gene of a target organism.

Infect, Infecting or Infection as used herein mean the ability of a virus to transfer its nucleic acid to a host or introduce a viral nucleic acid into a host, wherein the viral nucleic acid is replicated, viral proteins are synthesized, and new viral particles assembled.

Local Spread as used herein means the ability of a virus or viral vector to move through the plasmodesmata from an infected cell to an adjacent cell. By this method, a virus that is replicating within a cell will spread copies of itself to several surrounding cells. Replication of virus in the adjacent cells will usually result in another round of local spread of virus to further adjacent cells.

mRNA as used herein means Messenger RNA, translated on ribosomes to produce proteins.

Non-viral as used herein means any RNA segment, which is not normally contained within the virus whose modification is exploited for effecting gene transfer and is therefore used synonymously with “exogenous”. Therefore, a gene derived from a different virus species than that modified is included within the meaning of the terms “non-viral” and “exogenous” for the purposes of describing the invention. For example, a non-viral gene as the term is used herein could include a gene derived from a bacterial virus, an animal virus, or a plant virus of a type distinguishable from the virus modified to effect transformation. In addition, a non-viral gene may be a structural gene derived from any prokaryotic or eukaryotic organism. It will be understood by those ordinarily skilled in the art that there may exist certain genes whose transfer does not result in obvious phenotypic modification of the host cell. A phenotypic modification may occur, for example, if the translation product of the non-viral gene is toxic to the host cell, is degraded or processed in a manner, which renders it non-functional or possesses structural features, which render it impossible for the host cell to translate in sufficient quantities to confer a detectable phenotype on the transformed cells. However, the invention does not depend upon any specific property of an RNA segment or gene being transferred. Therefore, the possible existence of RNA segments or genes which fail to confer a readily observable phenotypic trait on recipient cells or plants is irrelevant to the invention and in any case will be readily recognizable by those of ordinary skill in the art without undue experimentation.

Phenotypic Trait as used herein means an observable, measurable or detectable property resulting from the expression or suppression of a gene or genes. Phenotype includes both easily observable traits and biochemical processes.

Plant Cell as used herein means the structural and physiological unit of plants, consisting of a protoplast and the cell wall.

Plant host as used herein means a cell, tissue or organism capable of replicating a nucleic acid such as a plant viral nucleic acid and which is capable of being infected by a virus containing the viral vector or viral nucleic acid. As used herein, plant host is intended to include whole plant, plant cell, and plant tissues, plant organ or plant part such as root, stem leaf, flower or seed.

Plant Organ as used herein means a distinct and visibly differentiated part of a plant, such as root, stem, leaf or embryo.

Plant Tissue as used herein means Any tissue of a plant in planta or in culture. This term is intended to include a whole plant, plant cell, plant organ, protoplast, cell culture, or any group of plant cells organized into a structural and functional unit.

(+)sense RNA (plus-sense RNA) as used herein means A virus with a single-stranded RNA genome of the same polarity (‘sense’) as mRNA.

Positive-sense Inhibition as used herein means A type of gene regulation based on inhibition of gene expression believed to be due to the presence in a cell of an RNA molecule substantially homologous to at least a portion of the mRNA being translated. The RNA molecule can be an exogenous coding sequence carried by an RNA viral vector of the type discussed herein.

Promoter as used herein means The 5′-flanking, non-coding sequence substantially adjacent a coding sequence which is involved in the initiation of transcription of the coding sequence.

Protoplast as used herein means an isolated plant cell without some or all of its cell wall.

Replication as used herein means Nucleic acid synthesis, the process of copying.

RNA virus as used herein means a virus whose genome is RNA in single-stranded form, the single strand being a (+) strand, or messenger-sense strand. Replication of the viral (+) strand in a virus-infected cell occurs by a process of direct RNA replication and is therefore distinguishable from the replication mechanism of retroviruses, which undergo an intermediate step of reverse transcription in the host cell.

Systemic Spread as used herein means the ability of a virus to spread from the vicinity of an initial infection to distant leaves. Systemic spread is accomplished by entry of the virus into the fluid circulation of the plant. From the point of entry, the virus is carried down to the roots and then up to other leaves.

Single component virus as used herein means a virus having a single nucleic acid sequence; unipartite. The single component virus is contrasted with the multicomponent virus, which has more than one nucleic acid component. Each component of a multicomponent virus is individually encapsidated, separate from the other(s).

Subgenomic Promoter as used herein means A promoter of a subgenomic mRNA of a viral nucleic acid. Plant viral nucleic acid can be modified to contain an exogenous nucleic acid sequence under the control of a subgenomic promoter.

Vector as used herein means an organism responsible for transmitting a pathogen from one host to another, e.g. a mosquito. (In molecular biology, a molecule used to clone nucleic acid sequences).

Viral Vector as used herein means a self-replicating RNA or DNA molecule derived from a virus which transfers an RNA or DNA segment between cells, such as bacteria, yeast, plant, or animal cells and contains an exogenous DNA or RNA segment to be expressed in the host.

Trans-acting replication element—In contrast to the single component (unipartite) virus, replication of a multipartite virus such as BMV depends upon the existence of one or more trans (diffusible) replication elements, which interact with a cis-acting replication element to carry out RNA replication. While trans-acting elements are necessary for replication of a multipartite virus such as BMV, they need not be present or coded for on the modified RNA provided they are made available within the infected cell by some other means. For example, in the case of a multipartite RNA virus, the trans-acting functions may be provided by other, unmodified components of the viral genome used to transform the cells simultaneously with the modified RNA. The target cell may also be modified in a previous step to provide constitutive expression of the trans-acting functions. In the case of a multipartite virus, the cis-acting element is therefore defined in functional terms: any modification which destroys the ability of the RNA to replicate in a cell known to contain the requisite trans-acting elements, is deemed to be a modification in the cis-acting replication element. Conversely, any modification, such as an insertion in a sequence region, which is able to tolerate such insertion without disrupting replication, is a modification outside the cis-acting replication element. At one time, it was commonly believed that all viruses contained a trans-acting replication factor. Today we know that many, and possibly all single component viruses do not have a trans-acting replication element.

A first embodiment demonstrates a capped viral vector having a single base inserted at the 5′ terminus of the viral sequence.

Another embodiment demonstrates a capped viral vector having a sequence of bases inserted at the 5′ terminus of the viral sequence.

In another embodiment, a host cell is infected by a capped viral vector which has a single base inserted at the 5′ terminus of the viral sequence. The capped viral vector is able to infect, to reproduce, to systemically infect the host plant, and to express an exogenous RNA segment.

In another embodiment, a host cell is infected by a capped viral vector having a sequence of bases inserted at the 5′ terminus of the viral sequence. The capped viral vector is able to infect the host cell, to reproduce, to systemically infect the host plant, and to express an exogenous RNA segment.

Another embodiment demonstrates an uncapped viral vector.

In another embodiment, an uncapped viral vector infects a host cell. The uncapped viral vector is able to reproduce, to systemically infect the host and to express an exogenous RNA segment.

Another embodiment demonstrates an uncapped viral vector having a single base inserted at the 5′ terminus of the viral sequence.

In another embodiment, an uncapped viral vector having a single base inserted at the 5′ terminus of the viral sequence infects a host cell. The uncapped viral vector is able to reproduce, to systemically infect the host and to express an exogenous RNA segment.

Another embodiment demonstrates an uncapped viral vector having a sequence of bases inserted at the 5′ terminus of the viral sequence.

In another embodiment, a host cell is infected by an uncapped viral vector having a sequence of bases inserted at the 5′ terminus of the viral sequence. The uncapped viral vector is able to reproduce, to systemically infect the host and to express an exogenous RNA segment.

An exogenous RNA segment may be inserted at any convenient insertion site provided the insertion does not disrupt a sequence essential for replication of the RNA within the host cell. For example, Dual Heterologous Subgenomic Promoter Expression System (DHSPES) in a plus stranded RNA vector has two subgenomic promoters. An exogenous RNA segment can be expressed in this system by inserting the exogenous gene at the 3′ end of one of the subgenomic promoters. This system is described in U.S. Pat. Nos. 5,316,931, 5,811,653, 5,589,367 and 5,866,785, the disclosure of which is incorporated by reference. An exogenous RNA segment under the control of a subgenomic promoter will be expressed in the host plant. Each heterologous subgenomic promoter is capable of transcribing or expressing adjacent genes or nucleic acid sequences in the plant host and incapable of recombination with each other and with native subgenomic promoters. One or more non-native nucleic acids may be inserted adjacent to the native plant viral subgenomic promoter or the native and non-native plant viral subgenomic promoters if more than one nucleic acid sequence is included. Moreover, it is specifically contemplated that two or more heterologous non-native subgenomic promoters may be used. The exogenous RNA segment may be transcribed or expressed in the host plant under the control of the subgenomic promoter to produce the products of the exogenous RNA segment.

A virus, whose coat protein is not essential for replication, an exogenous RNA segment may be inserted within or substituted for the region, which normally codes for coat protein. As desired, regions which contribute to undesirable host cell responses may be deleted or inactivated, provided such changes do not adversely effect the ability of the RNA to be replicated in the host cell. For many single component viruses, a reduction in the rate of normal RNA replication is tolerable and will in some instances be preferred, since the amount of RNA produced in a normal infection is more than enough to saturate the ribosomes of the transformed cell.

The transformation process itself can be carried out by any means whereby RNA can be introduced into cells, whole plants, plant tissues or protoplasts. The RNA alone or encapsidated in a virus particle can infect host cells, except that the modified viral RNA containing a non-viral RNA segment is substituted for its counterpart in a normal infection. Any other suitable means for introducing RNA into target cells such as microinjection may be used. Other variables of the infection process, such as pretreatment of the recipients, use of encapsidated or unencapsidated RNA, are matters of choice which those of ordinary skill in the art will be able to manipulate to achieve desired transformation efficiency in a given situation. For instance, the choice of single component plant RNA virus to be modified to achieve gene expression in a given plant variety will depend upon known host range properties of single component plant RNA viruses. For example, TMV infects a variety of Nicotiana species and their related domesticated relatives.

Plant cells, which are infected in culture, will normally remain transformed as the cells grow and divide since the RNA components are able to replicate and thus become distributed to daughter cells upon cell division. Plants regenerated from phenotypically modified cells, tissues or protoplasts remain phenotypically modified. Similarly, plants transformed as seedlings remain transformed during growth. Timing of application of the transforming components will be governed by the result that is intended and by variations in susceptibility to the transforming virus or viral RNA during various stages of plant growth.

Using the various embodiments of the invention, an exogenous segment RNA sequence can be expressed in a host by adapting the invention to any of a variety of embodiments set forth below for expressing an exogenous RNA segment. In one embodiment, an exogenous RNA segment is introduced into a plant host by way of a viral nucleic acid which comprises a native plant viral subgenomic promoter, a plant viral coat protein coding sequence, and at least one exogenous RNA segment under the control of a non-native subgenomic promoter.

In another embodiment, plant viral nucleic acid sequences used in the method of the present invention are characterized by the deletion of the native coat protein coding sequence in favor of a non-native plant viral coat protein coding sequence for the purpose of increasing host range. A non-native promoter, which could be the subgenomic promoter of the non-native coat protein coding sequence, controls expression of the non-native coat protein coding sequence. The non-native coat protein coding sequence is capable of expression in the plant host, of packaging the recombinant plant viral nucleic acid, and ensuring a systemic infection of a permissive host by the recombinant plant viral nucleic acid. The recombinant plant viral nucleic acid may contain one or more additional native or non-native subgenomic promoters.

In another embodiment, plant viral nucleic acids are used in the present invention wherein the native coat protein coding sequence is placed adjacent to a non-native subgenomic promoter.

In another embodiment, plant viral nucleic acids are used in the present invention wherein the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the product of the non-native nucleic acid. Alternatively, a non-native coat protein coding sequence may replace the native coat protein coding sequence.

In another embodiment, a viral expression vector contains an exogenous RNA segment encoding a polyprotein. At least one protein of the polyprotein is non-native to the vector. The non-native protein is released from the polyprotein by proteolytic processing catalyzed by at least one protease in the polyprotein. The vector comprises: a) at least one promoter; b) cDNA having a sequence, which codes for at least one polyprotein from a polyprotein-producing virus; c) at least one unique restriction site flanking a 3′ terminus of the cDNA; and a cloning vehicle.

Additional embodiments use a viral expression vector encoding at least one protein non-native to the vector that is released from at least one polyprotein expressed by the vector by proteolytic processing. The vector comprises at least one promoter, cDNA having a sequence which codes for at least one polyprotein from a polyprotein-producing virus, may contain at least one restriction site flanking a 3′ terminus of said cDNA and a cloning vehicle. Preferred embodiments include using a potyvirus as the polyprotein-producing virus, and especially preferred embodiments may use TEV (tobacco etch virus). A more detailed description of such vectors useful according to the method of the present invention may be found in U.S. Pat. Nos. 5,491,076 and 5,766,885 to James C. Carrington et al., which are incorporated herein by reference.

In yet other embodiments, recombinant plant viruses are used, which encode for the expression of a fusion between a plant viral coat protein and the amino acid product of the exogenous RNA of interest. Such a recombinant plant virus provides for a high level of expression of a nucleic acid of interest. The location or locations where the viral coat protein is joined to the amino acid product of the nucleic acid of interest may be referred to as the fusion joint. A given product of such a construct may have one or more fusion joints. The fusion joint may be located at the carboxyl terminus of the viral coat protein or the fusion joint may be located at the amino terminus of the coat protein portion of the construct. In instances where the nucleic acid of interest is located internal with respect to the 5′ and 3′ residues of the nucleic acid sequence encoding for the viral coat protein, there are two fusion joints. That is, the nucleic acid of interest may be located 5′, 3′, upstream, downstream or within the coat protein. In some embodiments of such recombinant plant viruses, a “leaky” start or stop codon may occur at a fusion joint, which sometimes does not result in translational termination. A more detailed description of some recombinant plant viruses according to this embodiment of the invention may be found in U.S. Pat. No. 5,977,438, the disclosure of which is incorporated herein by reference.

In another embodiment an exogenous gene can be introduced into the site of the coat protein gene of Potato virus X. Alternatively, an exogenous gene can be added to the PVX genome by partial duplication of the viral genome, so that expression of the exogenous gene is under the control of the same promoter sequence that controls production of the coat protein gene. Chapman, S. et al., The Plant Journal (1992) 2(4): 549-557.

Those skilled in the art will understand that these embodiments are representative only of many constructs, which may be useful to produce localized or systemic expression of nucleic acids in host organisms such as plants. All such constructs are contemplated and intended to be within the scope of the present invention.

The following examples illustrate the principles of the invention as applied to modification of TMV and the use of modified TMV containing a gene coding for green fluorescent protein (GFP) in the phenotypic transformation of Nicotiana plants and protoplasts. The following examples utilize many techniques well known and accessible to those skilled in the arts of molecular biology, cloning, plant cell biology, plant virology and plant tissue culture. Such methods are fully described in one or more of the cited references if not described in detail herein. Unless specified otherwise, enzymes were obtained from commercial sources and were used according to the vendor's recommendations or other variations known to the art. Those in the art also know reagents, buffers and culture conditions and reaction conditions for various enzyme-catalyzed reactions. Reference works containing such standard techniques include the following: R. Wu, ed. (1979) Meth. Enzymol. 68; R. Wu et al., eds. (1983) Meth. Enzymol. 100, 101; L. Grossman and K. Moldave, eds. (1980) Meth. Enzymol. 65; J. H. Miller (1972) Experiment's in Molecular Genetics; R. Davis et al. (1980) Advanced Bacterial Genetics; R. F. Schleif and P. C. Wensink (1982) Practical Methods in Molecular Biology; and T. Maniatis et al. (1982) Molecular Cloning.

Textual use of the name of a restriction endonuclease in isolation, e.g., “EcoRV” or “SphI” refers to use of that enzyme in an enzymatic digestion, except in a diagram where it can refer to the site of a sequence susceptible to action of that enzyme, e.g., a restriction site. In the text, restriction sites are indicated by the additional use of the word “site”, e.g., “EcoRV site”. The additional use of the word “fragment”, indicates a linear double-stranded DNA molecule having ends generated by action of the named enzyme (e.g., a restriction fragment). A phrase such as, “EcoRV/SphI” “fragment” indicates that the restriction fragment was generated by the action of two different enzymes, here EcoRV and SphI, the two ends resulting from the action of different enzymes. Note that the ends will have the characteristics of being either sticky (i.e., having a single strand of protrusion capable of base pairing with a complementary single-stranded oligonucleotide) or blunt (i.e., having no single-stranded protrusion). The specificity of a sticky end will be determined by the sequence of nucleotides comprising the single-stranded protrusion, which in turn is determined by the specificity of the enzyme, which produces it.

All plasmids are designated by a sequence of letters and numbers prefaced by a lower case “p”, for example, pBTI1037, pBTI1056, pBTI1057, pBTI SBS60, pBTI SBS60-29, or pBTI1056-GTN 28. Certain steps of cloning, selection and vector increase employed strains of E. Coli. While the strains used herein have been designated, there are many equivalent strains, available to the public that may be employed. The use of a particular microorganism as a substitute for a strain designated herein is a matter of routine choice available to those of ordinary skill in the art, according to well-known principles.

### EXAMPLES

### Example 1

### Infectivity of Uncapped and Capped Transcripts

This example demonstrates the production of highly infectious viral vector transcripts containing 5′ nucleotides with reference to the virus vector.

Insertion of base or base sequence at the 5′ end of the TMV cDNA.

Nucleotides were added between the transcriptional start site for in vitro transcription, in this case the T7 promoter, and the start of the cDNA of TMV in order to maximize transcription product yield and possibly obviate the need to cap virus transcripts to insure infectivity. The relevant sequence is the T7 promoter indicated in shorthand as TATA, followed by the transcription start site “G”, followed by TATTTT . . . , which is the continuation of the cDNA of TMV. These are put together as . . . TATAG TATTT . . . (SEQ ID NO: 1). The base preceding the “ ” is the start site for transcription of the cDNA. The bolded letter is the first base followed by TATTTT . . . of the TMV cDNA. Three approaches were taken:

1) addition of G, GG or GGG between the start site of transcription and the first base of the cDNA (as in . . . TATAG GTATTTT . . . and associated sequences);

| | | (SEQ ID NO:1) |
|---|---|---|
| I. | starting point | ...TATAG TATTTT... |
| | | (SEQ ID NO:2) |
| II. | addition of G | ...TATAG GTATTTT... |
| | | (SEQ ID NO:3) |
| III. | addition of GG | ...TATAG GGTATTTT... |
| | | (SEQ ID NO:4) |

| | | |
|---|---|---|
| IV. | addition of GGG | ...TATAG GGGTATTTT... |

2) addition of G and a random base (GN). As used herein, [N=A, T, C, or G]. VI represents addition of two random bases (N2). VII represents a G and two random bases (GNN). VIII represents three random bases (N3) between the start site of transcription and the TMV cDNA.

(SEQ ID NO:5)

| | | |
|---|---|---|
| V. | addition of GN | ...TATAG NGTATTTT... |

(SEQ ID NO:6)

| | | |
|---|---|---|
| VI. | addition of N2 | ...TATAGN NNTATTTT... |

(SEQ ID NO:7)

| | | |
|---|---|---|
| VII. | addition of GNN | ...TATAG NNGTATTTT... |

(SEQ ID NO:8)

| | | |
|---|---|---|
| VIII. | addition of NNN | ...TATAGN NNTATTTT... |

(SEQ ID NO:9)

| | | |
|---|---|---|
| IX. | addition of GNG | ...TATAGG NGTATTT... |

3) addition of a GT and a single random base (GTN) between the start site of transcription and the TMV cDNA (. . . TATAG TNGTATTTT, SEQ ID NO: 10 . . . and associated sequences).

| | | | |
|---|---|---|---|
| X. | addition of GTN | ...TATAG TNGTATTTT... | (SEQ ID NO:11) |
| XI. | addition of GTC, | ...TATAG TCGTATTTT... | (SEQ ID NO:12) |
| XII. | addition of (GTN).sub.2 | ...TATAG TNGTNGTATTTT... | (SEQ ID NO:13) |
| XIII. | addition of (GTN).sub.4 | ...TATAG TNGTNGTNGTNGTATTTT... | (SEQ ID NO:14) |
| XIV. | addition of GTATTT | ...TATAG TATTTGTATTTT,... | (SEQ ID NO:15) |

The use of random bases was based on the hypothesis that a particular base may be best suited for an additional nucleotide attached to the cDNA, since it will be complementary to the normal non-templated base incorporated at the 3′-end of the TMV (−) strand RNA. This allows for more ready mis-initiation and restoration of wild type sequence. The GTN would allow the mimicking of two potential sites for initiation, the added and the native sequence, and facilitate more ready mis-initiation of transcription in vivo to restore the native TMV cDNA sequence. Approaches included cloning GFP expressing TMV vector sequences into vectors containing:

1) an extra G,

2) an extra GG or

3) an extra GGG bases using standard molecular biology techniques.

Likewise, full length PCR of TMV expression clone 1056 was done to add

4) N2,

5) N3 and

6) GTN bases between the T7 promoter and the TMV cDNA.

Construction of Plasmid

DNA oligonucleotide primers were synthesized to contain a 5′ EcoRV site, an entire T7 RNA polymerase promoter, any extra nucleotides, and the 5′-terminal 20 bases of the TMV cDNA. These primers contain in the position for extra nucleotides, either none for constructs with sequence . . . TATAG TATTT . . . , a “G” for constructs with sequence . . . TATAG GTATTT . . . , a “GN” for constructs with sequence . . . TATAG NGTATTT . . . or a “GTN” for constructs with sequence . . . TATAG TNGTATTT . . . , where indicates the base preceding is the start site for transcription.

Examples of 5′ primers used to construct variant TMV constructs:

| | |
|---|---|
| 5′ GGCGATATCTAATACGACTCACTATAGTNGTAT | (SEQ ID NO:16) |
| TTTTACAACAATTACC | |
| 5′ GGCGATATCTAATACGACTCACTATAGNGTATT | (SEQ ID NO:17) |
| TTTACAACAATTACC | |
| 5′ GGCGATATCTAATACGACTCACTATAGNNGTAT | (SEQ ID NO:18) |
| TTTTACAACAATTACC | |
| 5′ GCGATATCTAATACGACTCACTATAGNNNGTAT | (SEQ ID NO:19) |
| TTTTACAACAATTACC | |
| 5′ GGCGATATCTAATACGACTCACTATAGTNGTNG | (SEQ ID NO:20) |
| TATTTTTACAACAATTAC | |

GATATC is the EcoRV restriction enzyme recognition site. Underlined is the T7 RNA polymerase promoter. The added bases between the T7 promoter and the TMV cDNA are in bold. The 5′ 20 bases of TMV cDNA are shown following the added bases.

We used the following 3′-primer, which anneals to TMV nucleotides 1034 to 1056:

5′ CACTATCTACACTTTTATGGGCC (SEQ ID NO: 21).

These 5′ primers and a 3′ primer containing sequences in the TMV cDNA surrounding the SphI site at position 445 were used to amplify a portion of the TMV cDNA (.about.500 bp in length) by the polymerase chain reaction (PCR). The PCR products were purified by agarose gel electrophoresis and standard gel extraction procedures and digested with EcoRV and SphI. The DNA fragments were then ligated into a plasmid digested with EcoRV and SphI. The digestion removed the identical portion of the genome and replaced it with the PCR fragment. DNA sequencing of the 5′ end of the TMV cDNA and T7 promoter junction analyzed by agarose gel electrophoresis and the recombinants. These plasmids were then used for in vitro transcription using T7 RNA polymerase.

In vitro Transcription

Several TMV-based virus expression vectors were initially used in these studies. Vector pBTI 1056 contains the T7 promoter (underlined) followed directly by the virus cDNA sequence (. . . TATAGTATT . . .), and vector pBTI SBS60-29 contains the T7 promoter followed by an extra guanine residue, then by the virus cDNA sequence (. . . TATAGGTATT . . .). Both expression vectors express an exogenous cycle 3, shuffled green fluorescent protein (GFPc3) in localized infection sites and systemically infected tissue of infected plants.

Transcriptions of each plasmid were carried out in the absence of cap analogue (uncapped) or in the presence of 8-fold greater concentration of RNA cap analogue than rGTP (capped). “r” means ribosomal.

Cap Transcriptions

1.2 .mu.l 20 mM rATP, rCTP, rUTP, 2 mM rGTP solution

2 .mu.l 10 mM RNA cap analogue (New England Biolabs catalog #1404, methylated cap analogue)

1 .mu.l Rnase Inhibitor 20 U (Promega N2511)

1 .mu.l T7 RNA polymerase 30 U (Ambion 2085)

2 .mu.l T7 RNA polymerase buffer (Ambion n supplied with enzyme)

0.5 mg of transcriptional plasmid DNA

Raise volume to 20 .mu.l

Incubate at 37.degree. C. for 1.5 hours

Analyze by agarose gel electrophoresis of 0.5 .mu.l solution.

Non-Cap Transcriptions

1.2 .mu.l 20 mM rATP, rCTP, rUTP

4.3 .mu.l 20 mM rGTP

1 .mu.l Rnase Inhibitor 20 U (Promega N2511)

1 .mu.l T7 RNA polymerase 30 U (Ambion 2085)

2 .mu.l T7 RNA polymerase buffer (Ambion, supplied with enzyme)

0.5 mg of transcriptional plasmid DNA

Raise volume to 20 .mu.l

Incubate at 37.degree. C. for 1.5 hours.

Analyze by agarose gel electrophoresis of 0.5 .mu.l solution.

There are other methods for transcription. This method is not intended to be limiting. The volume of rGTP is also not limiting. Other volumes can be used. While methylated cap is used in these experiments, for purposes of this invention, unmethylated cap, New England Biolabs catalog #1407, may also be used if cap is desired.

Description of Vectors pBTI SBS5, pBTI 1056, pBTI SBS60, pBTI SBS60-29, and pBTI 1056 GTN-28

Vector p30BGFPc3 is the base vector or starting point. Each clone comparison is outlined below. pBTI SBS5, pSBS60 and p1056 are compared with p3OBGFPc3. P1056GTN-28 is compared with p1056 and pSBS60-29 s compared with pSBS60. “nt” means nucleotide. “aa” means amino acid.

1. pBTI SBS5 (pBTI 1057) SEQ DATA vs pBTI 30BGFPc3
(Pbti 1037)

| 8 nt changes | | 4 aa changes |
|---|---|---|
| nt 1138 pBTI SB | S5 A to G mutation | (E to G change of aa 357 |
| | | of 126K protein) |
| nt 1268 | T to C | (silent) |
| nt 2382 pBTI SBS5 | A to G mutation | (K to E change of aa 772 |
| | | of 126K protein) |
| nt 3120 | T to C mutation | (silent) |
| nt 3632 pBTI SBS5 | G to A mutation | (silent) |
| nt 5213 | C to T mutation | (T to I change of aa 104 |
| | | of 30K protein) |
| nt 5303 pBTI SBS5 | A to G mutation | (K to R change of aa 134 |
| | | of 30K protein) |
| nt 5896 | C to A mutation | (silent) |

2. pBTI SBS60 SEQ DATA vs. pBTI 30BGFPc3 (pBTI 1037)

| 6 nt changes | | 1 aa change |
|---|---|---|
| nt 1268 | T to C | (silent) |
| nt 3120 | T to C mutation | (silent) |
| nt 4100 pBTI SBS60 | T to C mutation | (silent) |
| nt 5213 | C to T mutation | (T to I change of aa |
| | | 104 of 30K protein, |
| | | shared with pBTI SBS5) |
| nt 5634 pBTI SBS60 | A to G mutation | (silent) |
| nt 5896 | C to A mutation | (silent) |

There is no nucleotide “nt” sequence inserted between the T7 promoter sequence and the 5′ most base of the TMV U1 cDNA to form (. . . TATAGTATTTT . . .). In the short hand used herein . . . TATA represents the T7 promoter, there is no base or sequence of bases inserted between the T7 promoter and the GTATTTT . . . represents the 5′ most bases of the TMV U1 cDNA.

3. pBTI 1056 SEQ DATA vs. pBTI 30BGFPc3 (pBTI 1037)

| | | |
|---|---|---|
| 2 nt changes | | 2 aa change |
| nt 5213 | C to T mutation | (T to I change of aa 104 of 30k) |
| nt 5402 | G to A mutation | (R to K change of aa 167 of 30k) |

There is no nt sequence inserted between the T7 promoter sequence and the 5′ most base of the TMV U1 cDNA to form (. . . TATAGTATTTT . . .). In the short hand used herein . . . TATA represents the T7 promoter, there is no base or sequence of bases inserted between the T7 promoter and the GTATTTT . . . represents the 5′ most bases of the TMV U1 cDNA. pBTI1056.

4. pBTI 1056 GTN-28 SEQ DATA vs. pBTI 1056 nt sequence is GTC inserted between the T7 promoter sequence and the 5′ most base of the TMV U1 cDNA to form (. . . TATAGTCGTATTTT . . .). In the short hand used herein . . . TATA represents the T7 promoter, GTC is the inserted sequence of nucleotides, and GTATTTT . . . represents the 5′ most bases of the TMV U1 cDNA

5. pBTI SBS 60-29 SEQ DATA vs. pBTI SBS60 nt G is inserted between the T7 promoter sequence and the 5′ most base of the TMV U1 cDNA to form (. . . TATAGGTATTTT . . .). In the short hand used herein . . . TATA represents the T7 promoter, G is the inserted nucleotide, and GTATTTT . . . represents the 5′ most bases of the TMV U1 cDNA.

Table 1 summarizes the vectors and host plants used in the following experiments; the nucleotide sequence of each vector which contains the T7 promoter and the start of the cDNA of TMV is listed in the Table.

TABLE 1

| Viral | Cap Host | Foreign Plant+, |
|---|---|---|
| Vector | 5′ nucleotide sequence | −Plant Gene tissue |
| pBTI 1056 | TATAGTATTTT | + and NB and GFPc3 leaf - |
| | | NB 30K |
| pBTI SBS60- | TATAGGTATTTT | + and NB and GFPc3 leaf - |
| 29 | | NB 30K |
| pBTI SBS60 | TATAGTATTTT | + and NB GFPc3 protoplasts |
| pBTI 1056- | TATAGTCGTATTTT | + and NB and GFPc3 leaf - |
| GTN28 | | NB 30K |

Data of Cap and Non-cap Transcriptions of pBTI1056 and PBTI SBS60-29.

Nicotiana tabacum plants were infected with either capped or uncapped transcriptions (as described above) of pBTI 1056 and pBTI SBS60-29. Transcriptions were mixed with abrasive and inoculated on expanded older leaves of a wild type Nicotiana benthamiana (Nb) plant and a Nb plant expressing a TMV U1 30 k movement protein transgene (Nb 30K). Four days post inoculation (dpi), long wave UV light was used to judge the number of infection sites on the inoculated leaves of the plants. Systemic, noninoculated tissues were monitored from 4 dpi on for appearance of systemic infection indicating vascular movement of the inoculated virus. Table 2 shows the results of one representative experiment. An extra G, . . . TATAG GTATTTT . . . is found to be well tolerated as an additional 5′ nucleotide on the 5′ end of TMV vector RNA transcripts. Both capped and uncapped transcripts are infectious. Extra guanine residues located between the T7 promoter and the first base of a virus cDNA as demonstrated by pBTISBS60-29 lead to an increased amount of RNA transcript.

TABLE 2

| | |
|---|---|
| Local | Systemic |
| infection sites | Infection |

| Construct | Nb | Nb 30K | Nb | Nb 30K |
|---|---|---|---|---|
| pBTI 1056 | | | | |
| Capped | 5 | 6 | yes | yes |
| Uncapped | 0 | 5 | no | yes |
| pBTI SBS60-29 | | | | |
| Capped | 6 | 6 | yes | yes |
| Uncapped | 1 | 5 | yes | yes |

Results of Cap and Non-cap Transcriptions of pBTI SBS60

Nicotiana tabacum protoplasts were infected with either capped or uncapped transcriptions (as described above) of pBTI SBS60 which contains the T7 promoter followed directly by the virus cDNA sequence (TATAGTATT . . .). This expression vector also expresses the GFPc3 gene in infected cells and tissues. Nicotiana tabacum protoplasts were transfected with 1 l of each transcription. Approximately 36 hours post infection transfected protoplasts were viewed under UV illumination and cells showing GFPc3 expression. Approximately 80% of cells transfected with the capped pBTI SBS60 transcripts showed GFP expression while 5% of cells transfected with uncapped transcripts showed GFP expression. These experiments were repeated with higher amounts of uncapped inoculum. In this case a higher proportion of cells, >30% were found to be infected at this time with uncapped transcripts, where >90% of cells infected with greater amounts of capped transcripts were scored infected.

Data of Cap and Non-cap Transcriptions of pBTI1056 GTN-28

TMV-based virus expression vector pBTI 1056 GTN-28 contains the T7 promoter (underlined) followed GTC bases (bold) then the virus cDNA sequence (. . . TATAGTCGTATT, SEQ ID NO: 10, . . .). This expression vector expresses the exogenous cycle 3 shuffled green fluorescent protein (GFPc3) in localized infection sites and systemically infected tissue of infected plants. This vector was transcribed in vitro in the presence (capped) and absence (uncapped) of cap analogue as described above. Transcriptions were mixed with abrasive and inoculated on expanded older leaves of a wild type Nicotiana benthamiana (Nb) plant and a Nb plant expressing a TMV U1 30k movement protein transgene (Nb 30K). Four days post inoculation (dpi) long wave UV light was used to judge the number of infection sites on the inoculated leaves of the plants. Systemic, non-inoculated tissues were monitored from 4 dpi on for appearance of systemic infection indicating vascular movement of the inoculated virus. Table 3 shows data from two representative experiments at 11 dpi.

TABLE 3

| | |
|---|---|
| Local | Systemic |
| infection sites | Infection |

| Construct | Nb | Nb 30K | Nb | Nb 30K |
|---|---|---|---|---|
| Experiment 1 | | | | |
| pBTI 1056 GTN-28 | | | | |
| Capped | 18 | 25 | yes | yes |
| Uncapped | 2 | 4 | yes | yes |
| Experiment 2 | | | | |
| pBTI 1056 GTN-28 | | | | |
| Capped | 8 | 12 | yes | yes |
| Uncapped | 3 | 7 | yes | yes |

Extra GTN such as GTC residues located between the T7 promoter and the first base of a virus cDNA (pBTI 1056 GTN-28) lead to increased amount of RNA transcript as predicted by previous work with phage polymerases. These polymerases tend to initiate more efficiently at . . . TATAGTNG or . . . TATAGTCG than . . . TATAG. This has an indirect effect on the relative infectivity of uncapped transcripts in that greater amounts are synthesized per reaction resulting in enhanced infectivity.

Discussion and Conclusions

The foregoing examples demonstrate that, contrary to the practiced art in scientific literature and in issued patents (Ahlquist et al., U.S. Pat. No. 5,500,360), uncapped transcripts for virus expression vectors are infective in both whole plants and in plant cells, however with much lower specific infectivity. Therefore, capping is not a prerequisite for establishing an infection of a virus expression vector in plants; capping just increases the efficiency of infection. This reduced efficiency can be overcome, to some extent, by providing excess in vitro transcription product in an infection reaction for plants or plant cells. These data further support the claims concerning the utility of uncapped transcripts to initiate infections by plant virus expression vectors and further demonstrates that the introduction of extra, non-viral nucleotides at the 5′-end of in vitro transcripts does not preclude infectivity of uncapped transcripts. We conclude that while many similarities between plant viruses can be cited, there are specific differences between the Brome mosaic virus and the Tobamovirus group, which provide specific advantages to using a single-component Tobamovirus-derived vector. The results also show that reduced efficiency can be overcome, to some extent, by using a transgenic host plant or transgenic host plant cell, which expresses one or more RNA binding viral proteins. The expression of the 30K movement protein of TMV in transgenic plants also has the unexpected effect of equalizing the relative specific infectivity of uncapped verses capped transcripts. The mechanism behind this effect is not fully understood.

Further modifications and improvements following and embodying the teachings and disclosures herein are deemed to be within the scope of the invention, as set forth in the appended claims.

Although the invention has been described with reference to the presently preferred embodiments, it should be understood that various modifications could be made without departing from the spirit of the invention. Further modifications and improvements following and embodying the teachings and disclosures herein are deemed to be within the scope of the invention, as set forth in the appended claims. All patent applications, publications and other documents cited herein are incorporated by reference in their entirety for all purposes.

What is claimed is:

1. A capped RNA molecule capable of replication in the cytoplasm of a host plant cell located in a host plant, comprising:
a) a cis-acting viral replication element obtained from a single-component (+) strand RNA tobamovirus, the plant virus capable of replication in the cytoplasm of a host plant cell located in a host plant;
b) a sequence of one to three intervening bases located between a cap and the 5′ terminnus of the viral sequence; and
c) an exogenous RNA segment capable of expressing its function in a host plant cell; the exogenous RNA segment being located in a region of said capped RNA molecule able to tolerate said exogenous RNA segment without disrupting replication of said capped RNA molecule, wherein said uncapped RNA molecule is capable of replication in the absence of a trans-acting viral replication element in the host plant cell.

2. A capped RNA molecule capable of local spread in a host plant, comprising:
a) a cis-acting viral replication element obtained from a single-component (+) strand RNA tobamovirus, the plant virus capable of spreading locally from cell to cell in a host plant;
b) a sequence of one to three intervening bases located between a cap and the 5′ terminus of the viral sequence; and
c) an exogenous RNA segment capable of expressing its function in a host plant cell; the exogenous RNA segment being located in a region of said capped RNA molecule able to tolerate said exogenous RNA segment without disrupting replication of said capped RNA molecule, wherein said uncapped RNA molecule is capable of replication in the absence of a trans-acting viral replication element in the host plant cell.

3. A capped RNA molecule capable systemic spread in a host plant, comprising:
a) a cis-acting viral replication element obtained from a single-component (+) strand RNA tobamovirus, the plant virus capable systemic spread in a host plant;
b) a sequence of one to three intervening bases located between a cap and the 5′ terminus of the viral sequence; and
c) an exogenous RNA segment capable of expressing its function in a host plant cell; the exogenous RNA segment being located in a region of said capped RNA molecule able to tolerate said exogenous RNA segment without disrupting replication of said capped RNA molecule, wherein said uncapped RNA molecule is capable of replication in the absence of a trans-acting viral replication element in the host plant cell.


SEQUENCE LISTING (27 sequences)

SEQ ID NO: 1 - DNA, 11 bp, Tobacco mosaic virus
tatagtatttt

SEQ ID NO: 2 - DNA, 12 bp, Tobacco mosaic virus
tataggtatttt

SEQ ID NO: 3 - DNA, 13 bp, Tobacco mosaic virus
tatagggtatttt

SEQ ID NO: 4 - DNA, 14 bp, Tobacco mosaic virus
tataggggtatttt

SEQ ID NO: 5 - DNA, 13 bp, Tobacco mosaic virus
  misc_feature: n= A, T, C or G
tatagngtatttt

SEQ ID NO: 6 - DNA, 14 bp, Tobacco mosaic virus
  misc_feature: N= A, T, C or G
tatagnnntatttt

SEQ ID NO: 7 - DNA, 10 bp, Tobacco mosaic virus
  misc_feature (6)..(7): N= A, T, C or G
tatagnngta

SEQ ID NO: 8 - DNA, 14 bp, Tobacco mosaic virus
  misc_feature (6)..(8): N= A, T, C or G
tatagnnntatttt

SEQ ID NO: 9 - DNA, 13 bp, Tobacco mosaic virus
  misc_feature (7)..(7): N= A, T, C or G
tataggngtattt

SEQ ID NO: 10 - DNA, 14 bp, Tobacco mosaic virus
  misc_feature (7)..(7): N= A, T, C or G
tatagtngtatttt

SEQ ID NO: 11 - DNA, 14 bp, Tobacco mosaic virus
  misc_feature (7)..(7): N= A, T, C or G
tatagtngtatttt

SEQ ID NO: 12 - DNA, 14 bp, Tobacco mosaic virus
tatagtcgtatttt

SEQ ID NO: 13 - DNA, 17 bp, Tobacco mosaic virus
  misc_feature (7)..(7): N= A, T, C or G
tatagtngtngtatttt

SEQ ID NO: 14 - DNA, 23 bp, Tobacco mosaic virus
  misc_feature (7)..(7): N= A, T, C or G
tatagtngtngtngtngtatttt

SEQ ID NO: 15 - DNA, 17 bp, Tobacco mosaic virus
tatagtatttgtatttt

SEQ ID NO: 16 - DNA, 49 bp, Tobacco mosaic virus
  misc_feature (29)..(29): N= A, T, C or G
ggcgatatctaatacgactcactatagtngtatttttacaacaattacc

SEQ ID NO: 17 - DNA, 48 bp, Tobacco mosaic virus
  misc_feature (28)..(28): N= A, T, C or G
ggcgatatctaatacgactcactatagngtatttttacaacaattacc

SEQ ID NO: 18 - DNA, 49 bp, Tobacco mosaic virus
  misc_feature (28)..(28): N= A, T, C or G
ggcgatatctaatacgactcactatagnngtatttttacaacaattacc

SEQ ID NO: 19 - DNA, 50 bp, Tobacco mosaic virus
  misc_feature (28)..(30): N= A, T, C or G
ggcgatatctaatacgactcactatagnnngtatttttacaacaattacc

SEQ ID NO: 20 - DNA, 50 bp, Tobacco mosaic virus
  misc_feature (29)..(29): N= A, T, C or G
ggcgatatctaatacgactcactatagtngtngtatttttacaacaatta

SEQ ID NO: 21 - DNA, 23 bp, Tobacco mosaic virus
cactatctacacttttatgggcc

SEQ ID NO: 22 - DNA, 7685 bp, Tobacco mosaic virus
gtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcttgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatgggccttcttgagtttgtaactgctgctgggattacacatggcatggatgagctctacaaataatgacactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaacaacggagaggttcgaatcctcccctaaccgcgggtagcggccca

SEQ ID NO: 23 - DNA, 7686 bp, Tobacco mosaic virus
gtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgttcacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttaccaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttgggggaatgagttttaaaaagaataatttaatcgatgatgattcggaggctactgtcgccgaatcggattcgttttaaatagatcttacagtatcactactccatctcagttcgtgttcttgtcattaattaaatggctagcaaaggagaagaacttttcactggagttgtcccaattcttgttgaattagatggtgatgttaatgggcacaaattttctgtcagtggagagggtgaaggtgatgctacatacggaaagcttacacttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgacactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaaacaacggagaggttcgaatcctcccctaaccgcgggtagcggccca

SEQ ID NO: 24 - DNA, 7687 bp, Tobacco mosaic virus
ggtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgttcacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttaccaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttgggggaatgagttttaaaaagaataatttaatcgatgatgattcggaggctactgtcgccgaatcggattcgttttaaatagatcttacagtatcactactccatctcagttcgtgttcttgtcattaattaaatggctagcaaaggagaagaacttttcactggagttgtcccaattcttgttgaattagatggtgatgttaatgggcacaaattttctgtcagtggagagggtgaaggtgatgctacatacggaaagcttacacttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgacactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaaacaacggagaggttcgaatcctcccctaaccgcgggtagcggccca

SEQ ID NO: 25 - DNA, 7685 bp, Tobacco mosaic virus
gtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcttgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttataaaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatgggccttcttgagtttgtaactgctgctgggattacacatggcatggatgagctctacaaataatgacactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaacaacggagaggttcgaatcctcccctaaccgcgggtagcggccca

SEQ ID NO: 26 - DNA, 7686 bp, Tobacco mosaic virus
gtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttggggattcatcatcagtcaattactggtttcccaaaatgagggatatggtcatcgtaccattattcgacatttctttggagactagtaagaggacgcgcaaggaagtcttagtgtccaaggatttcgtgttcacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgagggagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcacctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgagaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaatggctagcaaaggagaagaacttttcactggagttgtcccaattcttgttgaattagatggtgatgttaatgggcacaaattttctgtcagtggagagggtgaaggtgatgctacatacggaaagcttacacttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgacactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaaacaacggagaggttcgaatcctcccctaaccgcgggtagcggccca

SEQ ID NO: 27 - DNA, 7688 bp, Tobacco mosaic virus
gtcgtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcttgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaagattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttataaaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaaatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatgggccttcttgagtttgtaactgctgctgggattacacatggcatggatgagctctacaaataatgacactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaacaacggagaggttcgaatcctcccctaaccgcgggtagcggccca